(19) United States
(12) Patent Application Publication
(10) Pub. No.: US 2021/0388106 A1
(43) Pub. Date: Dec. 16, 2021

(54) HEAVY CHAIN ANTIBODIES BINDING TO CD38

(71) Applicant: TeneoBio, Inc., Newark, CA (US)

(72) Inventors: Wim van Schooten, Newark, CA (US); Starlynn Clarke, Newark, CA (US); Kevin Dang, Newark, CA (US)

(21) Appl. No.: 17/288,485

(22) PCT Filed: Oct. 28, 2019
(86) PCT No.: PCT/US2019/058325

§ 371 Date: Apr. 23, 2021

Related U.S. Application Data
(60) Provisional application No. 62/751,520, filed on Oct. 26, 2018.

(51) Int. Cl. C07K 16/28

(52) U.S. Cl. CPC C07K 16/2896; C07K 16/2809; C07K 2317/76; C07K 2317/21; C07K 2317/31

(57) ABSTRACT

Binding compounds, such as human heavy-chain antibodies (e.g., UniAbs™) binding to CD38 are disclosed, along with methods of making such binding compounds, compositions, including pharmaceutical compositions, comprising such binding compounds, and their various uses.

### CROSS REFERENCE TO RELATED APPLICATIONS

This application claims priority benefit of the filing date of U.S. Provisional Patent Application No. 62/751,520, filed on Oct. 26, 2018, the disclosure of which application is herein incorporated by reference in its entirety.

### FIELD OF THE INVENTION

The present invention concerns binding compounds, such as human heavy-chain antibodies (e.g., UniAbs™) binding to CD38. Aspects of the invention relate to anti-CD38 heavy chain antibodies, combinations, including synergistic combinations, of anti-CD38 heavy chain antibodies targeting non-overlapping epitopes on CD38, multi-specific anti-CD38 heavy chain antibodies with binding specificity to more than one non-overlapping epitope on CD38, as well as methods of making such binding compounds, compositions, including pharmaceutical compositions, comprising such binding compounds, and their various uses.

### BACKGROUND OF THE INVENTION

### CD38 Ectoenzyme

The CD38 ectoenzyme is a membrane protein that has its catalytic site on the outside of the membrane in the extracellular compartment. This cell surface protein facilitates many functions and is found on a wide variety of cells, such as immune cells, endothelial cells, and neuronal tissue cells.

CD38, also known as ADP-ribosyl cyclase/cyclic ADP-ribose hydrolase 1, is a single-pass type II transmembrane protein with ectoenzymatic activities. Using NAD(P) as a substrate, it catalyzes the formation of several products: cyclic ADP-ribose (cADPR); ADP-ribose (ADPR); nicotinic acid adenine dinucleotide phosphate (NAADP); nicotinic acid (NA); ADP-ribose-2′-phosphate (ADPRP) (see, e.g. H. C. Lee, Mol. Med., 2006, 12: 317-323). CD38 can also use Nicotinamide Mononucleotide (NMN) as a substrate and convert it to nicotinamide and R5P (Liu et al., “Covalent and noncovalent intermediates of an NAD utilizing enzyme, human CD38.” Chem Biol 15(10): 1068-78.

CD38 is expressed predominantly on immune cells, including plasma cells, activated effector T cells, antigen-presenting cells, smooth muscle cells in the lung, Multiple Myeloma (MM) cells, B cell lymphoma, B cell leukemia cells, T cell lymphoma cells, breast cancer cells, myeloid derived suppressor cells, B regulatory cells, and T regulatory cells. CD38 on immune cells interacts with CD31/PECAM-1 expressed by endothelial cells and other cell lineages. This interaction promotes leukocyte proliferation, migration, T cell activation, and monocyte-derived DC maturation.

Antibodies binding to CD38 are described, for example, in Deckert et al., Clin. Cancer Res., 2014, 20(17):4574-83 and U.S. Pat. Nos. 8,153,765; 8,263,746; 8,362,211; 8,926,969; 9,187,565; 9,193,799; 9,249,226; and 9,676,869.

Daratumumab, an antibody specific for human CD38, was approved for human use in 2015 for the treatment of Multiple Myeloma (reviewed in Shallis et al., Cancer Immunol. Immunother. 2017, 66(6):697-703). Another antibody against CD38, Isatuximab (SAR650984), is in clinical trials for the treatment of Multiple Myeloma. (See, e.g., Deckert et al., Clin Cencer Res, 2014, 20(17):4574-83; Martin et al., Blood, 2015, 126:509; Martin et al., Blood, 2017, 129:3294-3303). These antibodies induce potent complement dependent cytotoxicity (CDC), antibody dependent cell-mediated cytotoxicity (ADCC), antibody dependent cellular phagocytosis (ADCP), and indirect apoptosis of tumor cells. Isatuximab also blocks the cyclase and hydrolase enzymatic activities of CD38 and induces direct apoptosis of tumor cells.

Examples of allosteric modulation of proteins by antibodies are human growth hormone, integrins, and beta-glactosidase (L. P. Roguin & L. A. Retegui, 2003, Scand. J. Immunol. 58(4):387-394). These examples show modulation of ligand-receptor interactions by single antibodies targeting different epitopes. One example of a bispecific antibody targeting two epitopes on a single molecule is against c-MET or hepatocyte growth factor receptor (HGFR) (DaSilva, J., Abstract 34: A MET×MET bispecific antibody that induces receptor degradation potently inhibits the growth of MET-addicted tumor xenografts. AACR Annual Meeting 2017; Apr. 1-5, 2017; Washington, D.C.).

### Heavy Chain Antibodies

In a conventional IgG antibody, the association of the heavy chain and light chain is due in part to a hydrophobic interaction between the light chain constant region and the CH1 constant domain of the heavy chain. There are additional residues in the heavy chain framework 2 (FR2) and framework 4 (FR4) regions that also contribute to this hydrophobic interaction between the heavy and light chains.

It is known, however, that sera of camelids (sub-order Tylopoda, which includes camels, dromedaries and llamas) contain a major type of antibodies composed solely of paired H-chains (heavy-chain only antibodies, heavy-chain antibodies, or UniAbs™). The UniAbs™ of Camelidae (Camelus dromedarius, Camelus bactrianus, Lama glama, Lama guanaco, Lama alpaca and Lama vicugna) have a unique structure consisting of a single variable domain (VHH), a hinge region and two constant domains (CH2 and CH3), which are highly homologous to the CH2 and CH3 domains of classical antibodies. These UniAbs™ lack the first domain of the constant region (CH1), which is present in the genome, but is spliced out during mRNA processing. The absence of the CH1 domain explains the absence of the light chain in the UniAbs™, since this domain is the anchoring place for the constant domain of the light chain Such UniAbs™ naturally evolved to confer antigen-binding specificity and high affinity by three CDRs from conventional antibodies, or fragments thereof (Muyldermans, 2001; J Biotechnol 74:277-302; Revets et al., 2005; Expert Opin Biol Ther 5:111-124). Cartilaginous fish, such as sharks, have also evolved a distinctive type of immunoglobulin, designated as IgNAR, which lacks the light polypeptide chains and is composed entirely by heavy chains IgNAR molecules can be manipulated by molecular engineering to produce the variable domain of a single heavy chain polypeptide (vNARs) (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

The ability of heavy chain-only antibodies devoid of light chain to bind antigen was established in the 1960s (Jaton et al. (1968) Biochemistry, 7, 4185-4195). Heavy chain immunoglobulin physically separated from light chain retained 80% of antigen-binding activity relative to the tetrameric antibody. Sitia et al. (1990) Cell, 60, 781-790 demonstrated that removal of the CH1 domain from a rearranged mouse μ gene results in the production of a heavy chain-only antibody, devoid of light chain, in mammalian cell culture. The antibodies produced retained VH binding specificity and effector functions.

Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)).

Mice in which the λ (lambda) light (L) chain locus and/or the λ and κ (kappa) L chain loci have been functionally silenced, and antibodies produced by such mice, are described in U.S. Pat. Nos. 7,541,513 and 8,367,888. Recombinant production of heavy chain-only antibodies in mice and rats has been reported, for example, in WO2006008548; U.S. Application Publication No. 20100122358; Nguyen et al., 2003, Immunology; 109(1), 93-101; Brüggemann et al., Crit. Rev. Immunol.; 2006, 26(5):377-90; and Zou et al., 2007, J Exp Med; 204(13): 3271-3283. The production of knockout rats via embryo microinjections of zinc-finger nucleases is described in Geurts et al., 2009, Science, 325(5939):433. Soluble heavy chain-only antibodies and transgenic rodents comprising a heterologous heavy chain locus producing such antibodies are described in U.S. Pat. Nos. 8,883,150 and 9,365,655. CAR-T structures comprising single-domain antibodies as a binding (targeting) domain are described, for example, in Iri-Sofia et al., 2011, Experimental Cell Research 317:2630-2641 and Jamnani et al., 2014, Biochim Biophys Acta, 1840:378-386.0

### SUMMARY OF THE INVENTION

Aspects of the invention include bispecific binding compounds comprising a first polypeptide having binding affinity to a first epitope on an ectoenzyme, and a second polypeptide having binding affinity to a second, non-overlapping epitope on the ectoenzyme. In some embodiments, the first polypeptide comprises an antigen-binding domain of a heavy-chain antibody having binding affinity to the first epitope. In some embodiments, the second polypeptide comprises an antigen-binding domain of a heavy-chain antibody having binding affinity to the second epitope. In some embodiments, the first and second polypeptides each comprise at least a portion of a hinge region. In some embodiments, the first and second polypeptides each comprise at least one CH domain. In some embodiments, the CH domain comprises a CH2 and/or a CH3 and/or a CH4 domain. In some embodiments, the CH domain comprises a CH2 domain and a CH3 domain. In some embodiments, the CH domain comprises a CH2 domain, a CH3 domain, and a CH4 domain. In some embodiments, the CH domain comprises a human IgG1 Fc region. In some embodiments, the human IgG1 Fc region is a silenced human IgG1 Fc region. In some embodiments, the CH domain comprises a human IgG4 Fc region. In some embodiments, the human IgG4 Fc region is a silenced human IgG4 Fc region. In some embodiments, the CH domain does not comprise a CH1 domain. In some embodiments, an asymmetric interface is present between the CH2 and/or the CH3 and/or the CH4 domains of the first and second polypeptides.

In some embodiments, the first polypeptide comprises a first antigen-binding domain of a heavy-chain antibody having binding affinity to the first epitope, and a second antigen-binding domain of a heavy-chain antibody having binding affinity to the second epitope. In some embodiments, the second polypeptide comprises a first antigen-binding domain of a heavy-chain antibody having binding affinity to the first epitope, and a second antigen-binding domain of a heavy-chain antibody having binding affinity to the second epitope. In some embodiments, the first and second antigen-binding domains are connected by a polypeptide linker. In some embodiments, the polypeptide linker consists of the sequence of SEQ ID NO: 45.

In some embodiments, a bispecific binding compound comprises a first and a second heavy chain polypeptide, each comprising an antigen-binding domain of a heavy-chain antibody having binding affinity to the first epitope, and a first and a second light chain polypeptide, each comprising an antigen-binding domain of a heavy-chain antibody having binding affinity to the second epitope. In some embodiments, the first and second light chain polypeptides each comprise a CL domain.

In some embodiments, the ectoenzyme is CD38.

Aspects of the invention include heavy-chain antibodies that bind to CD38 and that comprise an antigen-binding domain comprising: (i) a CDR1 sequence having two or fewer substitutions in any of the amino acid sequences of SEQ ID NOs: 1-5; and/or (ii) a CDR2 sequence having two or fewer substitutions in any of the amino acid sequences of SEQ ID NOs: 6-12; and/or (iii) a CDR3 sequence having two or fewer substitutions in any of the amino acid sequences of SEQ ID NOs: 13-17. In some embodiments, the CDR1, CDR2, and CDR3 sequences are present in a human framework. In some embodiments, a heavy-chain antibody further comprises a heavy chain constant region sequence in the absence of a CH1 sequence.

In some embodiments, a heavy-chain antibody comprises a variable region sequence having at least 95% sequence identity to any of the sequences of SEQ ID NOs: 18-28. In some embodiments, a heavy-chain antibody comprises a variable region sequence selected from the group consisting of SEQ ID NOs: 18-28. In some embodiments, a heavy-chain antibody is monospecific. In some embodiments, a heavy-chain antibody is multi-specific. In some embodiments, a heavy-chain antibody is bispecific. In some embodiments, a heavy-chain antibody has binding affinity to two different epitopes on the same CD38 protein. In some embodiments, the two different epitopes are non-overlapping epitopes. In some embodiments, a heavy-chain antibody has binding affinity to an effector cell. In some embodiments, a heavy-chain antibody has binding affinity to a T-cell antigen. In some embodiments, a heavy-chain antibody has binding affinity to CD3. In some embodiments, a heavy-chain antibody is in a CAR-T format.

Aspects of the invention include pharmaceutical compositions comprising a binding compound or a heavy-chain antibody described herein.

Aspects of the invention include therapeutic combinations comprising a binding compound or a heavy-chain antibody described herein and a second antibody that binds to CD38. In some embodiments, the second antibody that binds to CD38 is isatuximab or daratumumab.

Aspects of the invention include methods for the treatment of a disorder characterized by expression of CD38, the methods comprising administering to a subject with said disorder a binding compound or a heavy-chain antibody, a pharmaceutical composition, and/or a therapeutic combination as described herein. In some embodiments, the disorder is characterized by a hydrolase enzymatic activity of CD38. In some embodiments, the disorder is colitis. In some embodiments, the disorder is multiple myeloma (MM). In some embodiments, the disorder is an autoimmune disorder. In some embodiments, the disorder is rheumatoid arthritis (RA). In some embodiments, the disorder is pemphigus vulgaris (PV). In some embodiments, the disorder is systemic lupus erythematosus (SLE). In some embodiments, the disorder is multiple sclerosis (MS), systemic sclerosis or fibrosis. In some embodiments, the disorder is an ischemic injury. In some embodiments, the ischemic injury is an ischemic brain injury, an ischemic cardiac injury, an ischemic gastro-intestinal injury, or an ischemic kidney injury. In some embodiments, a method further comprises administering to the subject a second antibody that binds to CD38. In some embodiments, the second antibody that binds to CD38 is isatuximab or daratumumab.

These and further aspects will be further explained in the rest of the disclosure, including the Examples.

### BRIEF DESCRIPTION OF THE DRAWINGS

FIG. 1, panels A-E provide CDR sequences, variable region sequences, V-Gene and J-Gene information, percent CD38 hydrolase inhibition activity, and cell binding MFI data for anti-CD38 binding compounds in the F11 family.

FIG. 2, panels A-D, provide CDR sequences, variable region sequences, V-Gene and J-Gene information, percent CD38 hydrolase inhibition activity, and cell binding MFI data for anti-CD38 binding compounds in the F12 family.

FIG. 3, panels A-B, provide CDR sequences, variable region sequences, V-Gene and J-Gene information, percent CD38 hydrolase inhibition activity, and cell binding MFI data for anti-CD38 binding compounds in the F13 family.

FIG. 4 provides sequence information for additional amino acid sequences in the application.

FIG. 5 provides sequence information for additional amino acid sequences in the application.

FIG. 6 shows a graph depicting cell binding data as a function of concentration for the noted binding compounds.

FIG. 7 shows a graph depicting cell-based hydrolase activity as a function of concentration for the noted binding compounds.

FIG. 8 shows a graph depicting enzyme inhibition of the hydrolase activity of CD38 by bivalent UniAbs™.

FIG. 9 shows a graph depicting enzyme inhibition of the hydrolase activity of CD38 by a mixture of either UniAbs™ CD38_F13A or CD38_F13B with Isatuximab.

FIG. 10 shows a graph depicting direct cytotoxicity of Daudi cells induced with binding compounds in accordance with embodiments of the invention.

FIG. 11 shows a schematic representation of two bivalent (Panels C and D) and two tetravalent (Panels A and B) UniAb™ formats in accordance with embodiments of the invention.

FIG. 12 shows a graph depicting enzyme inhibition of the hydrolase activity of human CD38 expressed on CHO cells by tetravalent UniAbs™ as described in FIG. 11.

FIG. 13 shows a graph depicting inhibition of mixtures of UniAbs with Isatuximab.

FIG. 14 shows a graph depicting inhibition of hydrolase activity of CD38 by mixtures of UniAbs.

FIG. 15 shows another graph depicting inhibition of hydrolase activity of CD38 by mixtures of UniAbs.

FIG. 16 shows a graph depicting cell-based hydrolase activity for two tetravalent, bispecific binding compounds in accordance with embodiments of the invention, as depicted in FIG. 11.

FIG. 17 shows a graph depicting cell-based hydrolase activity for various binding compounds in accordance with embodiments of the invention.

FIG. 18 provides data in tabular format, summarizing various activities of binding compounds in accordance with embodiments of the invention.

FIG. 19, Panels A and B, show graphs depicting intracellular NAD+ concentration as a function of binding compound for Daudi and Ramos cells, respectively.

FIG. 20, Panels A-C, depict graphs showing results from T cell proliferation assays and IFNγ production assays.

FIG. 21 shows a graph depicting CD38 cyclase activity as a function of binding compound concentration for various binding compounds in accordance with embodiments of the invention.

FIG. 22 shows a graph depicting on target cell binding activity in three different cell lines as a function of binding compound concentration.

FIG. 23 shows a graph depicting off target cell binding activity in four different cell lines as a function of binding compound concentration.

FIG. 24, Panels A and B, show graphs depicting percent cell viability as a function of binding compound concentration for Daudi and Ramos cell lines, respectively. Panel C provides data in tabular format.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The terms “binding compound” and “binding composition” as used interchangeably herein refer to a molecular entity having binding affinity to one or more binding targets. Binding compounds in accordance with embodiments of the invention can include, without limitation, antibodies, antigen-binding domains of antibodies, antigen-binding fragments of antibodies, antibody-like molecules, heavy-chain antibodies (e.g., UniAbs™), ligands, receptors, and the like.

The term “antibody” is used herein in the broadest sense and specifically covers monoclonal antibodies, polyclonal antibodies, monomers, dimers, multimers, multispecific antibodies (e.g., bispecific antibodies), heavy-chain only antibodies, three chain antibodies, single chain Fv (scFv), nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule, or an immunologically active portion of any of these polypeptides, i.e., a polypeptide that comprises an antigen-binding site that immunospecifically binds an antigen of a target of interest or part thereof, such targets including but not limited to, cancer cells or cells that produce autoimmune antibodies associated with an autoimmune disease. The immunoglobulins disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. Light chains of the subject antibodies can be kappa light chains (Vkappa) or lambda light chains (Vlambda). The immunoglobulins can be derived from any species. In one aspect, the immunoglobulin is of largely human origin.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present invention can be made by the hybridoma method first described by Kohler et al. (1975) Nature 256:495, and can also be made via recombinant protein production methods (see, e.g., U.S. Pat. No. 4,816,567), for example.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen binding. The hypervariable region generally comprises amino acid residues from a “complementarity determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Exemplary CDR designations are shown herein, however, one of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlanet al. “Identification of specificity-determining residues in antibodies.” Faseb J. 1995; 9:133-139, each of which is herein specifically incorporated by reference.

The terms “heavy chain-only antibody,” and “heavy-chain antibody” are used interchangeably herein and refer, in the broadest sense, to antibodies lacking the light chain of a conventional antibody. The terms specifically include, without limitation, homodimeric antibodies comprising the VH antigen-binding domain and the CH2 and CH3 constant domains, in the absence of the CH1 domain; functional (antigen-binding) variants of such antibodies, soluble VH variants, Ig-NAR comprising a homodimer of one variable domain (V-NAR) and five C-like constant domains (C-NAR) and functional fragments thereof; and soluble single domain antibodies (sUniDabs™). In one embodiment, a heavy chain-only antibody is composed of the variable region antigen-binding domain composed of framework 1, CDR1, framework 2, CDR2, framework 3, CDR3, and framework 4. In another embodiment, the heavy chain-only antibody is composed of an antigen-binding domain, at least part of a hinge region and CH2 and CH3 domains, the absence of a CH1 domain. In another embodiment, the heavy chain-only antibody is composed of an antigen-binding domain, at least part of a hinge region and a CH2 domain. In a further embodiment, the heavy chain-only antibody is composed of an antigen-binding domain, at least part of a hinge region and a CH3 domain. Heavy chain-only antibodies in which the CH2 and/or CH3 domain is truncated are also included herein. In a further embodiment, the heavy chain is composed of an antigen binding domain, and at least one CH (CH1, CH2, CH3, or CH4) domain but no hinge region. In a further embodiment the heavy chain is composed of an antigen binding domain, at least one CH (CH1, CH2, CH3, or CH4) domain, and at least a portion of a hinge region. The heavy chain-only antibody can be in the form of a dimer, in which two heavy chains are disulfide bonded or otherwise, covalently or non-covalently, attached with each other. The heavy chain-only antibody may belong to the IgG subclass, but antibodies belonging to other subclasses, such as IgM, IgA, IgD and IgE subclass, are also included herein. In a particular embodiment, the heavy-chain antibody is of the IgG1, IgG2, IgG3, or IgG4 subtype, in particular the IgG1 or IgG4 subtype. In one embodiment, the heavy-chain antibody is of the IgG4 subtype, wherein one or more of the CH domains are modified to alter an effector function of the antibody. In one embodiment, the heavy-chain antibody is of the IgG1 subtype, wherein one or more of the CH domains are modified to alter an effector function of the antibody. Modifications of CH domains that alter effector function are further described herein. Non-limiting examples of heavy-chain antibodies are described, for example, in WO2018/039180, the disclosure of which is incorporated herein by reference in its entirety.

In one embodiment, the heavy chain-only antibodies herein are used as a binding (targeting) domain of a chimeric antigen receptor (CAR). The definition specifically includes human heavy chain-only antibodies produced by human immunoglobulin transgenic rats (UniRat™), called UniAbs™. The variable regions (VH) of UniAbs™ are called UniDabs™, and are versatile building blocks that can be linked to Fc regions or serum albumin for the development of novel therapeutics with multi-specificity, increased potency and extended half-life. Since the homodimeric UniAbs™ lack a light chain and thus a VL domain, the antigen is recognized by one single domain, i.e., the variable domain (antigen-binding domain) of the heavy chain of a heavy-chain antibody (VH).

An “intact antibody chain” as used herein is one comprising a full length variable region and a full length constant region (Fc). An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called κ (kappa) and λ (lambda), based on the amino acid sequences of their constant domains. Antibodies in accordance with embodiments of the invention can comprise kappa light chain sequences or lambda light chain sequences.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIIB1; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). The human IgG1 amino acid sequence (UniProtKB No. P01857) is provided herein as SEQ ID NO: 43. The human IgG4 amino acid sequence (UniProtKB No. P01861) is provided herein as SEQ ID NO: 44. Silenced IgG1 is described, for example, in Boesch, A. W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of a binding compound can comprise one or more mutations in the hinge region to eliminate disulfide bonding. In yet another embodiment, the hinge region of an Fc can be removed entirely. In still another embodiment, a binding compound can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The Fc may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms, e.g., Pichia pastoris, and mammalian cell lines, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al, BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See, e.g., WO 07/117505). The presence of particular glycosylation moieties has been shown to alter the function of immunoglobulins. For example, the removal of sugar chains from an Fc molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See, e.g., Kaneko, et al, Science 313:760 (2006)), whereas removal of fucose from the IgG leads to enhanced ADCC activity (See, e.g., Shoj-Hosaka, et al, J. Biochem., 140:777 (2006)).

In alternative embodiments, binding compounds of the invention may have an Fc sequence with enhanced effector functions, e.g., by increasing their binding capacities to FcγRIIIA and increasing ADCC activity. For example, fucose attached to the N-linked glycan at Asn-297 of Fc sterically hinders the interaction of Fc with FcγRIIIA, and removal of fucose by glyco-engineering can increase the binding to FcγRIIIA, which translates into >50-fold higher ADCC activity compared with wild type IgG1 controls. Protein engineering, through amino acid mutations in the Fc portion of IgG1, has generated multiple variants that increase the affinity of Fc binding to FcγRIIIA. Notably, the triple alanine mutant S298A/E333A/K334A displays 2-fold increase binding to FcγRIIIA and ADCC function. S239D/I332E (2×) and S239D/I332E/A330L (3×) variants have a significant increase in binding affinity to FcγRIIIA and augmentation of ADCC capacity in vitro and in vivo. Other Fc variants identified by yeast display also showed the improved binding to FcγRIIIA and enhanced tumor cell killing in mouse xenograft models. See, e.g., Liu et al. (2014) JBC 289(6):3571-90, herein specifically incorporated by reference.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

“Humanized” forms of non-human (e.g., rodent) antibodies, including single chain antibodies, are chimeric antibodies (including single chain antibodies) that contain minimal sequence derived from non-human immunoglobulin. See, e.g., Jones et al, (1986) Nature 321:522-525; Chothia et al (1989) Nature 342:877; Riechmann et al (1992) J. Mol. Biol. 224, 487-499; Foote and Winter, (1992) J. Mol. Biol. 224:487-499; Presta et al (1993) J. Immunol. 151, 2623-2632; Werther et al (1996) J. Immunol. Methods 157:4986-4995; and Presta et al (2001) Thromb. Haemost. 85:379-389. For further details, see U.S. Pat. Nos. 5,225,539; 6,548,640; 6,982,321; 5,585,089; 5,693,761; 6,407,213; Jones et al (1986) Nature, 321:522-525; and Riechmann et al (1988) Nature 332:323-329.

Aspects of the invention include binding compounds having multi-specific configurations, which include, without limitation, bispecific, trispecific, etc. A large variety of methods and protein configurations are known and used in bispecific monoclonal antibodies (BsMAB), tri-specific antibodies, etc.

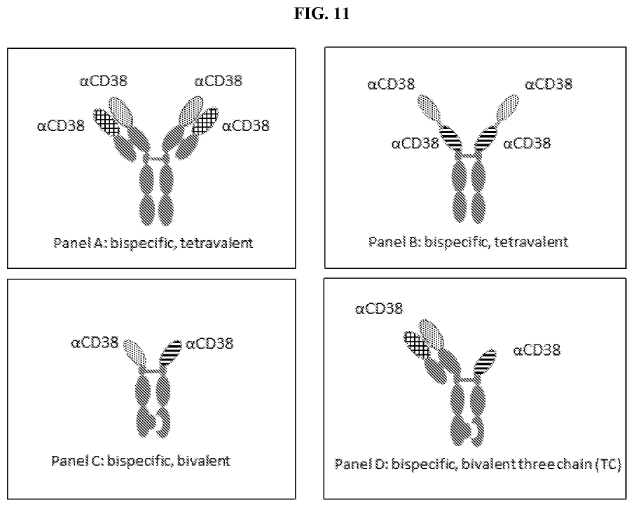

Aspects of the invention include antibodies comprising a heavy chain-only variable region in a monovalent or bivalent configuration. As used herein, the term “monovalent configuration” as used in reference to a heavy chain-only variable region domain means that only one heavy chain-only variable region domain is present, having a single binding site (see, e.g., FIG. 11, Panel D, right side of depicted molecule). In contrast, the term “bivalent configuration” as used in reference to a heavy chain-only variable region domain means that two heavy chain-only variable region domains are present (each having a single binding site), and are connected by a linker sequence (see, e.g., FIG. 11, Panel B, either side of depicted molecule). Non-limiting examples of linker sequences are discussed further herein, and include, without limitation, GS linker sequences of various lengths. When a heavy chain-only variable region is in a bivalent configuration, each of the two heavy chain-only variable region domains can have binding affinity to the same antigen, or to different antigens (e.g., to different epitopes on the same protein; to two different proteins, etc.). However, unless specifically noted otherwise, a heavy chain-only variable region denoted as being in a “bivalent configuration” is understood to contain two identical heavy chain-only variable region domains, connected by a linker sequence, wherein each of the two identical heavy chain-only variable region domains have binding affinity to the same target antigen.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 29) (n=1) and GGGGSGGGGS (SEQ ID NO: 45) (n=2). Other suitable linkers can also be used, and are described, for example, in Chen et al., Adv Drug Deliv Rev. 2013 Oct. 15; 65(10): 1357-69, the disclosure of which is incorporated herein by reference in its entirety.

The term “bispecific three-chain antibody like molecule” or “TCA” is used herein to refer to antibody-like molecules comprising, consisting essentially of, or consisting of three polypeptide subunits, two of which comprise, consist essentially of, or consist of one heavy and one light chain of a monoclonal antibody, or functional antigen-binding fragments of such antibody chains, comprising an antigen-binding region and at least one CH domain. This heavy chain/light chain pair has binding specificity for a first antigen. In some embodiments, a TCA comprises a light chain polypeptide subunit comprising a CDR1 sequence of SEQ ID NO: 49, a CDR2 sequence of SEQ ID NO: 50, and a CDR3 sequence of SEQ ID NO: 51, in a human light chain framework. In some embodiments, the human light chain framework is a human kappa (Vkappa) or a human lambda (Vlambda) framework. In some embodiments, a TCA comprises a light chain polypeptide subunit comprising a light chain variable region (VL) comprising a sequence having at least about 80%, 85%, 90%, 95%, or 99% identity to the sequence of SEQ ID NO: 52. In some embodiments, a TCA comprises a light chain polypeptide subunit that comprises the sequence of SEQ ID NO: 52. In some embodiments, a TCA comprises a light chain polypeptide subunit that comprises a light chain constant region (CL). In some embodiments, the light chain constant region is a human kappa light chain constant region or a human lambda light chain constant region. In some embodiments, a TCA comprises a light chain polypeptide subunit comprising a full length light chain comprising a sequence having at least about 80%, 85%, 90%, 95%, or 99% identity to the sequence of SEQ ID NO: 48. In some embodiments, a TCA comprises a light chain polypeptide subunit that comprises the sequence of SEQ ID NO: 48. The third polypeptide subunit comprises, consists essentially of, or consists of a heavy-chain only antibody comprising an Fc portion comprising CH2 and/or CH3 and/or CH4 domains, in the absence of a CH1 domain, and an antigen binding domain that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments.

A TCA binding compound makes use of a “heavy chain only antibody” or “heavy chain antibody” or “heavy chain polypeptide” which, as used herein, mean a single chain antibody comprising heavy chain constant regions CH2 and/or CH3 and/or CH4 but no CH1 domain. In one embodiment, the heavy chain antibody is composed of an antigen-binding domain, at least part of a hinge region and CH2 and CH3 domains. In another embodiment, the heavy chain antibody is composed of an antigen-binding domain, at least part of a hinge region and a CH2 domain. In a further embodiment, the heavy chain antibody is composed of an antigen-binding domain, at least part of a hinge region and a CH3 domain. Heavy chain antibodies in which the CH2 and/or CH3 domain is truncated are also included herein. In a further embodiment the heavy chain is composed of an antigen binding domain, and at least one CH (CH1, CH2, CH3, or CH4) domain but no hinge region. The heavy chain only antibody can be in the form of a dimer, in which two heavy chains are disulfide bonded or otherwise covalently or non-covalently attached with each other, and can optionally include an asymmetric interface between two or more of the CH domains to facilitate proper pairing between polypeptide chains. The heavy-chain antibody may belong to the IgG subclass, but antibodies belonging to other subclasses, such as IgM, IgA, IgD and IgE subclass, are also included herein. In a particular embodiment, the heavy chain antibody is of the IgG1, IgG2, IgG3, or IgG4 subtype, in particular the IgG1 subtype or the IgG4 subtype. Non-limiting examples of a TCA binding compound are described in, for example, WO2017/223111 and WO2018/052503, the disclosures of which are incorporated herein by reference in their entirety.

Heavy-chain antibodies constitute about one fourth of the IgG antibodies produced by the camelids, e.g., camels and Ilamas (Hamers-Casterman C., et al. Nature. 363, 446-448 (1993)). These antibodies are formed by two heavy chains but are devoid of light chains. As a consequence, the variable antigen binding part is referred to as the VHH domain and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

The term “interface”, as used herein, is used to refer to a region, which comprises those “contact” amino acid residues (or other non-amino acid groups such as, for example, carbohydrate groups,) in a first heavy chain constant region which interact with one or more “contact” amino acid residues (or other non-amino acid groups) in a second heavy chain constant region.

The term “asymmetric interface” is used to refer to an interface (as hereinabove defined) formed between two polypeptide chains, such as a first and a second heavy chain constant region and/or between a heavy chain constant region and its matching light chain, wherein the contact residues in the first and the second chains are different by design, comprising complementary contact residues. The asymmetric interface can be created by, e.g., knobs/holes interactions and/or salt bridges coupling (charge swaps) and/or other techniques known in the art.

A “cavity” or “hole” refers to at least one amino acid side chain which is recessed from the interface of the second polypeptide and therefore accommodates a corresponding protuberance (“knob”) on the adjacent interface of the first polypeptide. The cavity (hole) may exist in the original interface or may be introduced synthetically (e.g., by altering a nucleic acid encoding the interface residue). Normally, a nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. The upper limit for the number of original residues which are replaced is the total number of residues in the interface of the second polypeptide. The preferred import residues for the formation of a cavity are usually naturally occurring amino acid residues and are preferably selected from alanine (A), serine (S), threonine (T), valine (V) and glycine (G). Most preferred amino acid residues are serine, alanine or threonine, most preferably alanine. In one preferred embodiment, the original residue for the formation of the protuberance has a large side chain volume, such as tyrosine (Y), arginine (R), phenylalanine (F) or tryptophan (W). Asymmetric interfaces are described in detail, for example, in Xu et al., “Production of bispecific antibodies in ‘knobs-into-holes’ using a cell-free expression system”, MAbs. 2015, 7(1):231-42, the disclosure of which is incorporated by reference herein in its entirety.

The term “CD38” as used herein refers to a single-pass type II transmembrane protein with ectoenzymatic activities, also known as ADP-ribosyl cyclase/cyclic ADP-ribose hydrolase 1. The term “CD38” includes a CD38 protein of any human or non-human animal species, and specifically includes human CD38 as well as CD38 of non-human mammals.

The term “human CD38” as used herein includes any variants, isoforms and species homologs of human CD38 (UniProt P28907), regardless of its source or mode of preparation. Thus, “human CD38” includes human CD38 naturally expressed by cells, and CD38 expressed on cells transfected with the human CD38 gene.

The terms “anti-CD38 heavy chain-only antibody,” “CD38 heavy chain-only antibody,” “anti-CD38 heavy-chain antibody” and “CD38 heavy-chain antibody” are used herein interchangeably to refer to a heavy chain-only antibody as hereinabove defined, immunospecifically binding to CD38, including human CD38, as hereinabove defined. The definition includes, without limitation, human heavy chain antibodies produced by transgenic animals, such as transgenic rats or transgenic mice expressing human immunoglobulin, including UniRats™ producing human anti-CD38 UniAb™ antibodies, as hereinabove defined.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly-available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” binding compound (such as an isolated antibody) is one which has been identified and separated and/or recovered from a component of its natural environment. Contaminant components of its natural environment are materials which would interfere with diagnostic or therapeutic uses for the binding compound, and may include enzymes, hormones, and other proteinaceous or non-proteinaceous solutes. In preferred embodiments, the binding compound will be purified (1) to greater than 95% by weight of binding compound as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated binding compound includes the binding compound in situ within recombinant cells, since at least one component of the binding compound's natural environment will not be present. Ordinarily, however, isolated binding compound will be prepared by at least one purification step.

Binding compounds in accordance with embodiments of the invention include multi-specific binding compounds. Multi-specific binding compounds have more than one binding specificity. The term “multi-specific” specifically includes “bispecific” and “trispecific,” as well as higher-order independent specific binding affinities, such as higher-order polyepitopic specificity, as well as tetravalent binding compounds and antigen-binding fragments of binding compounds (e.g., antibodies and antibody fragments). “Multi-specific” binding compounds specifically include antibodies comprising a combination of different binding entities as well as antibodies comprising more than one of the same binding entity. The terms “multi-specific antibody,” “multi-specific heavy chain-only antibody,” “multi-specific heavy-chain antibody,” and “multi-specific UniAb™” are used herein in the broadest sense and cover all antibodies with more than one binding specificity. The multi-specific heavy chain anti-CD38 antibodies of the present invention specifically include antibodies immunospecifically binding to two or more non-overlapping epitopes on a CD38 protein, such as a human CD38.

An “epitope” is the site on the surface of an antigen molecule to which an antigen-binding region of a binding compound binds. Generally, an antigen has several or many different epitopes, and reacts with many different binding compounds (e.g., many different antibodies). The term specifically includes linear epitopes and conformational epitopes.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

“Polyepitopic specificity” refers to the ability to specifically bind to two or more different epitopes on the same or different target(s). As noted above, the present invention specifically includes anti-CD38 heavy-chain antibodies with polyepitopic specificities, i.e., anti-CD38 heavy-chain antibodies binding to two or more non-overlapping epitopes on a CD38 protein, such as a human CD38. The term “non-overlapping epitope(s)” or “non-competitive epitope(s)” of an antigen is defined herein to mean epitope(s) that are recognized by one member of a pair of antigen-specific antibodies, but not the other member. Pairs of antibodies, or antigen-binding regions targeting the same antigen on a multi-specific antibody, recognizing non-overlapping epitopes, do not compete for binding to that antigen and are able to bind that antigen simultaneously.

A binding compound binds “essentially the same epitope” as a reference binding compound (e.g., a reference antibody), when the binding compound and the reference antibody recognize identical or sterically overlapping epitopes. The most widely used and rapid methods for determining whether two epitopes bind to identical or sterically overlapping epitopes are competition assays, which can be configured in all number of different formats, using either labeled antigen or labeled antibody. Usually, the antigen is immobilized on a 96-well plate, and the ability of unlabeled antibodies to block the binding of labeled antibodies is measured using radioactive or enzyme labels.

The term “compete” as used herein with respect to a binding compound (e.g., an antibody) and a reference binding compound (e.g., a reference antibody) means that the binding compound causes about a 15-100% reduction in the binding of the reference binding compound to the target antigen, as determined by standard techniques, such as by the competition binding assays described herein.

The term “competition group” as used herein refers to two or more binding compounds (e.g., a first and a second antibody) that bind to the same target antigen (or epitope) and that compete with the members of the competition group for binding to the target antigen. Members of the same competition group compete with one another for binding to a target antigen, but do not necessarily have the same functional activity.

The term “valent” as used herein refers to a specified number of binding sites in an antibody molecule or binding compound.

A “multi-valent” binding compound has two or more binding sites. Thus, the terms “bivalent”, “trivalent”, and “tetravalent” refer to the presence of two binding sites, three binding sites, and four binding sites, respectively. Thus, a bispecific antibody according to the invention is at least bivalent and may be trivalent, tetravalent, or otherwise multi-valent. A large variety of methods and protein configurations are known and used for the preparation of bispecific monoclonal antibodies (BsMAB), tri-specific antibodies, and the like.

The term “chimeric antigen receptor” or “CAR” is used herein in the broadest sense to refer to an engineered receptor, which grafts a desired binding specificity (e.g., the antigen-binding region of a monoclonal antibody or other ligand) to membrane-spanning and intracellular-signaling domains. Typically, the receptor is used to graft the specificity of a monoclonal antibody onto a T cell to create a chimeric antigen receptor (CAR). (Dai et al., J Natl Cancer Inst, 2016; 108(7):djv439; and Jackson et al., Nature Reviews Clinical Oncology, 2016; 13:370-383.).

The term “human antibody” is used herein to include antibodies having variable and constant regions derived from human germline immunoglobulin sequences. The human antibodies herein may include amino acid residues not encoded by human germline immunoglobulin sequences, e.g., mutations introduced by random or site-specific mutagenesis in vitro or by somatic mutation in vivo. The term “human antibody” specifically includes heavy chain-only antibodies having human heavy chain variable region sequences, produced by transgenic animals, such as transgenic rats or mice, in particular UniAbs™ produced by UniRats™, as defined above.

By a “chimeric antibody” or a “chimeric immunoglobulin” is meant an immunoglobulin molecule comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the invention that have been engineered to produce such chimeric antibodies.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell, such as a natural killer cell, is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T cell receptors or FcRs and perform effector functions. Preferably, the cells express at least FcγRIII and perform ADCC effector function. Examples of human leukocytes which mediate ADCC include natural killer (NK) cells, monocytes, cytotoxic T cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B cells and T cells including cytolytic T cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods 202:163 (1996), may be performed.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, Calif.) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12(8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “B-cell neoplasms” or “mature B-cell neoplasms” in the context of the present invention include, but are not limited to, all lymphoid leukemias and lymphomas, chronic lymphocytic leukemia, acute lymphoblastc leukemia, prolymphocytic leukemia, precursor B-lymphoblastic leukemia, hair cell leukemia, small lymphocytic lymphoma, B-cell prolymphocytic lymphoma, B-cell chronic lymphocytic leukemia, mantle cell lymphoma, Burkitt's lymphoma, follicular lymphoma, diffuse large B-cell lymphoma (DLBCL), multiple myeloma, lymphoplasmacytic lymphoma, splenic marginal zone lymphoma, plasma cell neoplasms, such as plasma cell myeloma, plasmacytoma, monoclonal immunoglobulin deposition disease, heavy chain disease, MALT lymphoma, nodal marginal B cell lymphoma, intravascular large B cell lymphoma, primary effusion lymphoma, lymphomatoid granulomatosis, non-Hodgkins lymphoma, Hodgkins lymphoma, hairy cell leukemia, primary effusion lymphoma and AIDS-related non-Hodgkins lymphoma.

The term “colitis” as used herein broadly refers to a disorder characterized by inflammation of the lining of the colon. As used herein, “colitis” includes autoimmune colitis, which can be caused by inflammatory bowel disease, ulcerative colitis, or Crohn's disease; treatment-induced colitis, such as diversion colitis, chemical colitis, chemotherapy-induced colitis, or colitis that is induced by treatment with one or more therapeutic agents, e.g., PD-1/PD-L1, CTLA-4, TIGIT, TIM-3, LAG-3, and other immune checkpoint inhibitors; vascular disease, such as ischemic colitis; infectious colitis, such as infectious colitis caused by Clostridium difficile, Shiga toxin, or parasitic infection (e.g., Entamoeba histolytica); colitis of unknown origin, e.g., microscopic colitis, lymphocytic colitis, or collagenous colitis; or atypical colitis (i.e., colitis that does not conform to criteria for clinically accepted types of colitis).

The term “ischemic injury” as used herein refers to any injury caused by diminished blood flow to a tissue. Ischemic injuries include, but are not limited to, ischemic brain injury, ischemic cardiac injury, ischemic kidney injury, ischemic gastro-intestinal (GI) injury, etc.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, and/or individuals with autoimmune diseases, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

The terms “synergy” and “synergistic” as used herein refer to a combination of two or more individual components (e.g., two or more heavy-chain antibodies) that are together more effective at achieving a particular result (e.g., a reduction in hydrolase activity) as compared to the results achieved when the two or more individual components are used separately. For example, a synergistic combination of two or more hydrolase blocking heavy-chain antibodies is more effective at inhibiting hydrolase activity than either of the individual hydrolase blocking heavy-chain antibodies when used separately. Similarly, a synergistic therapeutic combination is more effective than the effects of the two or more single agents that make up the therapeutic combination. A determination of a synergistic interaction between two or more single agents in a therapeutic combination can be based on results obtained from various assays known in the art. The results of these assays can be analyzed using the Chou and Talalay combination method and Dose-Effect Analysis with CalcuSyn software in order to obtain a Combination Index “CI” (Chou and Talalay (1984) Adv. Enzyme Regul. 22:27-55). A combination therapy may provide “synergy” and prove “synergistic”, i.e., the effect achieved when the active ingredients used together is greater than the effects that result from using the compounds separately. A synergistic effect may be attained when the active ingredients are: (1) co-formulated and administered or delivered simultaneously in a combined, unit dosage formulation; (2) delivered by alternation as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially in time.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomeriation), clipping/hydrolysis/fragmentation (e.g., hinge region fragmentation), succinimide formation, unpaired cysteine(s), N-terminal extension, C-terminal processing, glycosylation differences, etc.

### II. Detailed Description

The invention is based, at least in part, on the finding that binding compounds, such as heavy-chain antibodies, that have binding specificity to one or more epitopes on an ectoenzyme can be used to lyse tumor cells and/or inhibit enzymatic activity of a target ectoenzyme. The invention is also based, at least in part, on the finding that binding compounds, or combinations thereof, that have binding specificity for at least two non-overlapping epitopes on an ectoenzyme (e.g., multispecific, e.g., bispecific binding compounds) work synergistically to lyse tumor cells and/or modulate (e.g., inhibit) enzymatic activity of the target ectoenzyme. Aspects of the invention therefore relate to binding compounds, including, without limitation, monospecific binding compounds having binding specificity for a single target (e.g., a single epitope on an ectoenzyme), as well as multispecific (e.g., bispecific) binding compounds having binding specificity for at least two targets (e.g., a first and a second epitope on an ectoenzyme). Aspects of the invention also relate to therapeutic combinations of the binding compounds described herein, as well as methods of making and using such binding compounds.

### Ectoenzymes

Ectoenzymes are a diverse group of membrane proteins having catalytic sites outside the plasma membrane. Many ectoenzymes are found on leukocytes and endothelial cells, where they play multiple biological roles. Apart from the extracellular catalytic activity that is common to all, ectoenzymes are a diverse class of molecules that are involved in very different types of enzymatic reactions. Different ectoenzymes can modulate each step of leukocyte—endothelial contact interactions, as well as subsequent cell migration in tissues. Ectoenzymes include, without limitation, CD38, CD10, CD13, CD26, CD39, CD73, CD156b, CD156c, CD157, CD203, VAP1, ART2, and MT1-MMP.

The ectoenzyme CD38 belongs to the family of nucleotide-metabolizing enzymes which, in addition to recycling nucleotides, generate compounds that control cellular homeostasis and metabolism. The catalytic activity of CD38 is required for various physiological processes, including insulin secretion, muscarinic Ca²⁺ signaling in pancreatic acinar cells, neutrophil chemotaxis, dendritic cell trafficking, oxytocin secretion, and in the development of diet-induced obesity. See, Vaisitti et al., Laeukemia, 2015, 29: 356-368, and the references cited therein. CD38 has bifunctional ecto-enzymatic cyclase as well as hydrolase activity. CD38 is expressed in a variety of malignancies, including chronic lymphocytic leukemia (CLL). CD38 has been shown to identify a particularly aggressive form of CLL, and is considered a negative prognostic marker, predicting a shorter overall survival of patients with this aggressive variant of CLL. See, Malavasi et al., 2011, Blood, 118:3470-3478, and Vaisitti, 2015, supra.

CD38 is also expressed on solid tumors, and is overexpressed on tumor cells of PD1-refractory non-small cell lung cancer patients (SNCLC) (Chen et al., Cancer Discov, 8(9): 1156-75). CD38 possibly plays a role in other solid tumors that are resistant to immune checkpoint blockade, such pancreatic tumors, renal cell carcinoma, melanoma, colo-rectal carcinoma, and others.

### Anti-CD38 Binding Compounds

Aspects of the invention include binding compounds having binding affinity to an ectoenzyme, such as CD38. The binding compounds can include, without limitation, a variety of antibody-like molecules, such as those depicted in FIG. 11. In some embodiments, a binding compound includes a variable domain of an antibody having binding affinity to a particular epitope on an ectoenzyme. In some embodiments, a binding compound includes at least one antigen-binding domain of a heavy-chain antibody having binding affinity to a particular epitope. In certain embodiments, a binding compound includes two or more antigen-binding domains, wherein one antigen-binding domain has binding affinity to a first epitope, and one antigen-binding domain has binding affinity to a second epitope. In certain embodiments, the epitopes are non-overlapping epitopes. The binding compounds described herein provide a number of benefits that contribute to utility as clinically therapeutic agent(s). The binding compounds include members with a range of binding affinities, allowing the selection of a specific sequence with a desired binding affinity.

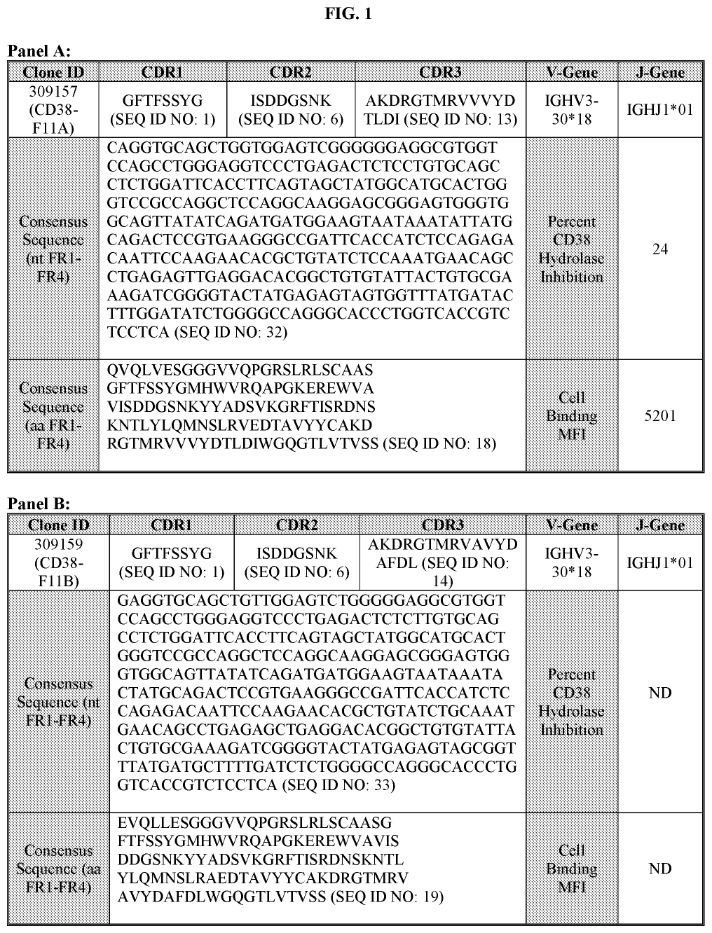

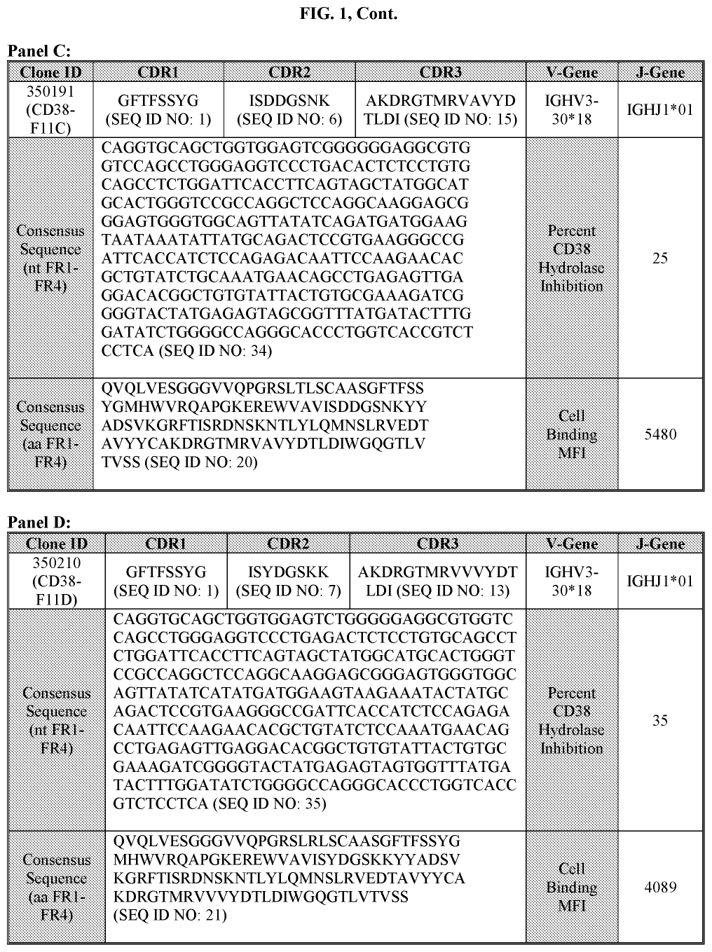

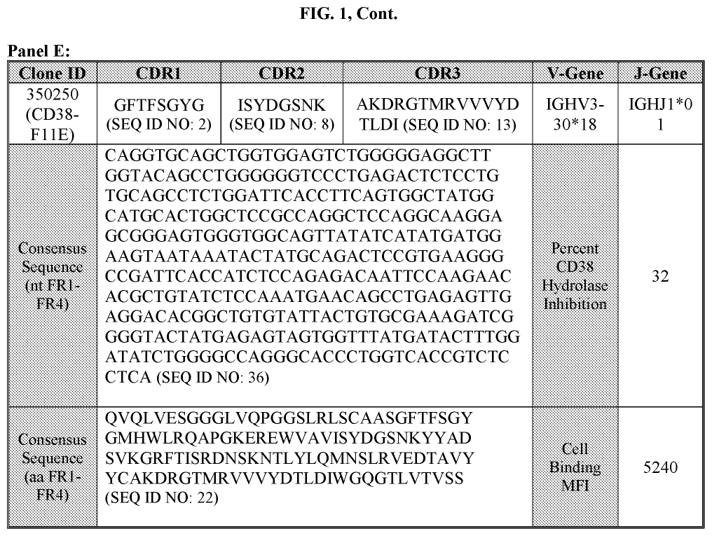

Aspects of the invention include heavy-chain antibodies that bind to human CD38. The antibodies comprise a set of CDR sequences as defined herein and shown in FIGS. 1-3 and 5, and are exemplified by the provided heavy chain variable region (VH) sequences of SEQ ID NOs: 18-28 set forth in FIGS. 1-3. The antibodies provide a number of benefits that contribute to utility as clinically therapeutic agent(s). The antibodies include members with a range of binding affinities, allowing the selection of a specific sequence with a desired binding affinity.

A suitable binding compound may be selected from those provided herein for development and therapeutic or other use, including, without limitation, use as a bispecific binding compound, e.g., as shown in FIG. 11, or a tri-specific antibody, or part of a CAR-T structure.

Determination of affinity for a candidate protein can be performed using methods known in the art, such as Biacore measurements. Binding compounds described herein may have an affinity for CD38 with a Kd of from about 10⁻⁶ to around about 10⁻¹¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating, e.g., blocking, a CD38 biological activity, such as hydrolase activity, including in vitro assays, pre-clinical models, and clinical trials, as well as assessment of potential toxicity.

The binding compounds described herein are not cross-reactive with the CD38 protein of Cynomolgus macaque, but can be engineered to provide cross-reactivity with the CD38 protein of Cynomolgus macaque, or with the CD38 of any other animal species, if desired.

The CD38-specific binding compounds herein comprise an antigen-binding domain, comprising CDR1, CDR2 and CDR3 sequences in a human VH framework. The CDR sequences may be situated, as an example, in the region of around amino acid residues 26-35; 53-59; and 98-117 for CDR1, CDR2 and CDR3, respectively, of the provided exemplary variable region sequences set forth in SEQ ID NOs: 18-28. It will be understood by one of ordinary skill in the art that the CDR sequences may be in different positions if a different framework sequence is selected, although generally the order of the sequences will remain the same.

Representative CDR1, CDR2 and CDR3 sequences are shown in FIGS. 1-3, and 5.

In some embodiments, an anti-CD38 heavy-chain antibody of the invention comprises a CDR1 sequence of any one of SEQ ID NOs: 1-5. In a particular embodiment, the CDR1 sequence is SEQ ID NO: 1. In a particular embodiment, the CDR1 sequence is SEQ ID NO: 3. In a particular embodiment, the CDR1 sequence is SEQ ID NO: 4.

In some embodiments, an anti-CD38 heavy-chain antibody of the invention comprises a CDR2 sequence of any one of SEQ ID NOs: 6-12. In a particular embodiment, the CDR2 sequence is SEQ ID NO: 6. In a particular embodiment, the CDR2 sequence is SEQ ID NO: 9. In a particular embodiment, the CDR2 sequence is SEQ ID NO: 11.

In some embodiments, an anti-CD38 heavy-chain antibody of the invention comprises a CDR3 sequence of any one of SEQ ID NOs: 13-17. In a particular embodiment, the CDR3 sequence is SEQ ID NO: 13. In a particular embodiment, the CDR3 sequence is SEQ ID NO: 16. In a particular embodiment, the CDR3 sequence is SEQ ID NO: 17.

In a further embodiment, an anti-CD38 heavy-chain antibody of the invention comprises the CDR1 sequence of SEQ ID NO: 1; the CDR2 sequence of SEQ ID NO: 6; and the CDR3 sequence of SEQ ID NO: 13. In a further embodiment, an anti-CD38 heavy-chain antibody of the invention comprises the CDR1 sequence of SEQ ID NO: 3; the CDR2 sequence of SEQ ID NO: 9; and the CDR3 sequence of SEQ ID NO: 16. In a further embodiment, an anti-CD38 heavy-chain antibody of the invention comprises the CDR1 sequence of SEQ ID NO: 4; the CDR2 sequence of SEQ ID NO: 11; and the CDR3 sequence of SEQ ID NO: 17.

In further embodiments, an anti-CD38 heavy-chain antibody of the invention comprises any of the heavy chain variable region amino acid sequences of SEQ ID NOs: 18-28 (FIGS. 1-3).

In a still further embodiment, an anti-CD38 heavy-chain antibody of the present invention comprises the heavy chain variable region sequence of SEQ ID NO: 18. In a still further embodiment, an anti-CD38 heavy-chain antibody of the present invention comprises the heavy chain variable region sequence of SEQ ID NO: 23. In a still further embodiment, an anti-CD38 heavy-chain antibody of the present invention comprises the heavy chain variable region sequence of SEQ ID NO: 27.

In some embodiments, a CDR sequence in an anti-CD38 heavy-chain antibody of the invention comprises one or two amino acid substitutions relative to a CDR1, CDR2 and/or CDR3 sequence or set of CDR1, CDR2 and CDR3 sequences in any one of SEQ ID NOs: 1-17 (FIGS. 1-3) or SEQ ID NOs: 49-51 (FIG. 5). In some embodiments, the heavy-chain anti-CD38 antibodies herein will comprise a heavy chain variable region sequence with at least about 85% identity, at least 90% identity, at least 95% identity, at least 98% identify, or at least 99% identity to any of the heavy chain variable region sequences of SEQ ID NOs: 18-28 (shown in FIGS. 1-3) or SEQ ID NOs: 46 or 47 (shown in FIG. 5).

In some embodiments, bispecific or multispecific binding compounds are provided, which may have any of the configurations discussed herein, including, without limitation, a bispecific, bivalent heavy-chain antibody comprising two non-identical polypeptide subunits that are associated with one another via an asymmetric interface; a bispecific, tetravent heavy-chain antibody comprising two identical polypeptide subunits, each containing a first and a second antigen-binding domain; a bispecific, tetravalent heavy-chain antibody comprising two identical heavy chain polypeptide subunits and two identical light chain polypeptide subunits; or a bispecific three-chain antibody-like molecule, comprising a first heavy chain polypeptide subunit, a first light chain polypeptide subunit, and a second heavy chain polypeptide subunit.

In some embodiments, a bispecific antibody can comprise at least one heavy chain variable region having binding specificity for CD38, and at least one heavy chain variable region having binding specificity for a protein other than CD38. In some embodiments, a bispecific antibody can comprise a heavy chain/light chain pair that has binding specificity for a first antigen, and a heavy chain from a heavy chain-only antibody, comprising an Fc portion comprising CH2 and/or CH3 and/or CH4 domains, in the absence of a CH1 domain, and an antigen binding domain that binds an epitope of a second antigen or a different epitope of the first antigen (e.g., a second, non-overlapping epitope on a CD38 protein). In one particular embodiment, a bispecific antibody comprises a heavy chain/light chain pair that has binding specificity for an antigen on an effector cell (e.g., a CD3 protein on a T cell), and a heavy chain from a heavy chain-only antibody comprising an antigen-binding domain that has binding specificity for CD38.

In some embodiments, where a protein of the invention is a bispecific antibody, one arm of the antibody (one binding moiety) is specific for human CD38, while the other arm may be specific for target cells, tumor-associated antigens, targeting antigens, e.g., integrins, etc., pathogen antigens, checkpoint proteins, and the like. Target cells specifically include cancer cells, including, without limitation, cells from hematologic tumors, e.g., B-cell tumors, as discussed below.

Various formats of bispecific binding compounds are within the ambit of the invention, including, without limitation, single chain polypeptides, two chain polypeptides, three chain polypeptides, four chain polypeptides, and multiples thereof. The bispecific binding compounds herein specifically include T cell bispecific antibodies binding to CD38, which is expressed predominantly on immune cells, and CD3 (anti-CD38×anti-CD3 antibodies). Such antibodies induce potent T cell mediated killing of cells expressing CD38.

In some embodiments, a binding compound includes a first and a second polypeptide, i.e., a first and a second polypeptide subunit, wherein each polypeptide comprises an antigen-binding domain of a heavy-chain antibody. In some embodiments, each of the first and second polypeptides further includes a hinge region, or at least a portion of a hinge region, which can facilitate formation of at least one disulfide bond between the first and second polypeptides. In some embodiments, each of the first and second polypeptides further includes at least one heavy chain constant region (CH) domain, such as a CH2 domain, and/or a CH3 domain, and/or a CH4 domain. In certain embodiments, the CH domain lacks a CH1 domain. The antigen-binding domain of each of the first and second polypeptides can incorporate any of the CDR sequences and/or variable region sequences described herein in order to impart antigen-binding capability on the binding compound. As such, in certain embodiments, each polypeptide in the binding compound can include an antigen-binding domain that has binding specificity to the same epitope, or to different epitopes (e.g., a first and a second, non-overlapping epitope on CD38 protein).

A non-limiting example of a binding compound in accordance with embodiments of the invention is depicted in FIG. 11, Panel C. In the depicted embodiment, the binding compound is a bispecific, bivalent heavy-chain antibody that comprises a first polypeptide comprising an antigen-binding domain of a heavy-chain antibody, at least a portion of a hinge region, a CH domain comprising a CH2 and a CH3 domain (and lacking a CH1 domain), and a second polypeptide comprising an antigen-binding domain of a heavy-chain antibody, at least a portion of a hinge region, and a CH domain comprising a CH2 and a CH3 domain (and lacking a CH1 domain) The depicted embodiment includes an asymmetric interface between the CH3 domain of the first polypeptide and the CH3 domain of the second polypeptide, and at least one disulfide bond in the hinge region that connects the first and second polypeptides to form the binding compound. Asymmetric interfaces in accordance with embodiments of the invention are further described herein.

In some embodiments, a binding compound includes a first and a second polypeptide, i.e., a first and a second polypeptide subunit, wherein each polypeptide comprises two antigen binding domains. In some embodiments, each of the first and second polypeptides further includes a hinge region, or at least a portion of a hinge region, which can facilitate formation of at least one disulfide bond between the first and second polypeptides. In some embodiments, each of the first and second polypeptides further includes at least one heavy chain constant region (CH) domain, such as a CH2 domain, and/or a CH3 domain, and/or a CH4 domain. In certain embodiments, the CH domain lacks a CH1 domain. The antigen-binding domain of each of the first and second polypeptides can incorporate any of the CDR sequences and/or variable region sequences described herein in order to impart antigen-binding capability on the binding compound. As such, in certain embodiments, each polypeptide in the binding compound can include two antigen-binding domains, having binding specificity to the same epitope, or to different epitopes (e.g., a first and a second, non-overlapping epitope on a CD38 protein).

A non-limiting example of a binding compound in accordance with embodiments of the invention is depicted in FIG. 11, Panel B. In the depicted embodiment, the binding compound is a bispecific, tetravalent binding compound that comprises a first polypeptide comprising two antigen-binding domains, one with binding specificity to a first epitope and one with binding specificity to a second, non-overlapping epitope, at least a portion of a hinge region, a CH domain comprising a CH2 and a CH3 domain (and lacking a CH1 domain), and a second polypeptide comprising two antigen-binding domains, one with binding specificity to the first epitope and one with binding specificity to the second, non-overlapping epitope, at least a portion of a hinge region, a CH domain comprising a CH2 and a CH3 domain (and lacking a CH1 domain). The depicted embodiment includes at least one disulfide bond in the hinge region that connects the first and second polypeptides to form the binding compound.

In some embodiments, the first and second antigen-binding domains on each polypeptide are connected by a polypeptide linker. One non-limiting example of a polypeptide linker that can connect the first and second antigen-binding domains is a GS linker, such as the G4S linker having the amino acid sequence GGGGS (SEQ ID NO: 29). Other suitable linkers can also be used, and are described, for example, in Chen et al., Adv Drug Deliv Rev. 2013 Oct. 15; 65(10: 1357-69, the disclosure of which is incorporated herein by reference in its entirety.

In some embodiments, a binding compound includes a first and a second heavy chain polypeptide, i.e., first and second heavy chain polypeptide subunits, as well as a first and a second light chain polypeptide, i.e., first and second light chain polypeptide subunits. In some embodiments, each of the heavy chain polypeptides comprises an antigen-binding domain of a heavy-chain antibody. In some embodiments, each of the heavy chain polypeptides further includes a hinge region, or at least a portion of a hinge region, which can facilitate formation of at least one disulfide bond between the first and second heavy chain polypeptides. In some embodiments, each of the first and second heavy chain polypeptides further includes at least one heavy chain constant region (CH) domain, such as a CH2 domain, and/or a CH3 domain, and/or a CH4 domain. In certain embodiments, the CH domain includes a CH1 domain. The antigen-binding domain of each of the first and second heavy chain polypeptides can incorporate any of the CDR sequences and/or variable region sequences described herein in order to impart antigen-binding capability on the binding compound.

In some embodiments, each of the light chain polypeptides comprises an antigen-binding domain of a heavy-chain antibody. In some embodiments, each of the light chain polypeptides further includes a light chain constant region (CL) domain. The antigen-binding domain of each of the first and second light chain polypeptides can incorporate any of the CDR sequences and/or variable region sequences described herein in order to impart antigen-binding capability on the binding compound. Additionally, the CH1 domains on the heavy chain polypeptides and the CL domains on the light chain polypeptides can each include at least one cysteine residue that facilitates formation of a disulfide bond that connects each light chain polypeptide to one of the heavy chain polypeptides.

A non-limiting example of a binding compound in accordance with embodiments of the invention is depicted in FIG. 11, Panel A. In the depicted embodiment, the binding compound is a bispecific, tetravalent binding compound comprising two heavy chain polypeptides and two light chain polypeptides. Each heavy chain polypeptide comprises an antigen-binding domain with binding specificity to a first epitope, a CH1 domain, at least a portion of a hinge region, a CH2 domain and a CH3 domain. The depicted embodiment includes at least one disulfide bond in the hinge region that connects the first and second heavy chain polypeptides. Each light chain polypeptide comprises an antigen-binding domain with binding specificity to a second epitope, and a CL domain. The depicted embodiment includes at least one disulfide bond between the CL and CH1 domains that connects the first and second heavy chain polypeptides to the first and second light chain polypeptides to form the binding compound.

A non-limiting example of a binding compound in accordance with embodiments of the invention is depicted in FIG. 11, Panel D. In the depicted embodiment, the binding compound is a bispecific, bivalent binding compound comprising three polypeptides (two heavy chain polypeptides and one light chain polypeptide). The first heavy chain polypeptite subunit and the light chain polypeptide subunit together form a binding unit having binding affinity to a first epitope, and the second heavy chain polypeptide comprises a heavy chain-only variable region having binding affinity to a second epitope. In some embodiments, the second polypeptide subunit comprises a single heavy chain-only variable region domain (monovalent configuration). In some embodiments, the second polypeptide subunit comprises two heavy chain-only variable regions (bivalent configuration), connected by a linker. The first heavy chain polypeptide comprises an antigen-binding domain with binding specificity to a first epitope, a CH1 domain, at least a portion of a hinge region, a CH2 domain and a CH3 domain. The depicted embodiment includes at least one disulfide bond in the hinge region that connects the first and second heavy chain polypeptides. The light chain polypeptide comprises an antigen-binding domain with binding specificity to the first epitope, and a CL domain.

In one preferred embodiment, a bispecific binding compound having binding affinity to a first CD38 epitope and a second, non-overlapping CD38 epitope comprises a first polypeptide having binding affinity to the first CD38 epitope comprising an antigen-binding domain of a heavy-chain antibody comprising a CDR1 sequence of SEQ ID NO: 1, a CDR2 sequence of SEQ ID NO: 6, and a CDR3 sequence of SEQ ID NO: 13, at least a portion of a hinge region, and a CH domain comprising a CH2 domain and a CH3 domain, and a second polypeptide having binding affinity to the second CD38 epitope comprising an antigen-binding domain of a heavy-chain antibody comprising a CDR1 sequence of SEQ ID NO: 3, a CDR2 sequence of SEQ ID NO: 9, and a CDR3 sequence of SEQ ID NO: 16, at least a portion of a hinge region, and a CH domain comprising a CH2 domain and a CH3 domain, and an asymmetric interface between the CH3 domain of the first polypeptide and the CH3 domain of the second polypeptide. In certain preferred embodiments, this binding compound comprises an Fc region that is a human IgG1 Fc region, a human IgG4 Fc region, a silenced human IgG1 Fc region, or a silenced human IgG4 Fc region.

In another preferred embodiment, a bispecific binding compound having binding affinity to a first CD38 epitope and a second, non-overlapping CD38 epitope includes two identical polypeptides, each polypeptide comprising a first antigen-binding domain of a heavy-chain antibody having binding affinity to the first CD38 epitope, comprising a CDR1 sequence of SEQ ID NO: 1, a CDR2 sequence of SEQ ID NO: 6, and a CDR3 sequence of SEQ ID NO: 13, a second antigen-binding domain of a heavy-chain antibody having binding affinity to the second CD38 epitope, comprising a CDR1 sequence of SEQ ID NO: 3, a CDR2 sequence of SEQ ID NO: 9, and a CDR3 sequence of SEQ ID NO: 16, at least a portion of a hinge region, and a CH domain comprising a CH2 domain and a CH3 domain. In certain preferred embodiments, this binding compound comprises an Fc region that is a human IgG1 Fc region, a human IgG4 Fc region, a silenced human IgG1 Fc region, or a silenced human IgG4 Fc region.

In another preferred embodiment, a bispecific binding compound having binding affinity to a first CD38 epitope and a second, non-overlapping CD38 epitope comprises a first and a second heavy chain polypeptide, each comprising an antigen-binding domain of a heavy-chain antibody having binding affinity to the first CD38 epitope, comprising a CDR1 sequence of SEQ ID NO: 1, a CDR2 sequence of SEQ ID NO: 6, and a CDR3 sequence of SEQ ID NO: 13, at least a portion of a hinge region, and a CH domain comprising a CH1 domain, a CH2 domain and a CH3 domain, and a first and a second light chain polypeptide, each comprising an antigen-binding domain of a heavy-chain antibody having binding affinity to the second CD38 epitope, comprising a CDR1 sequence of SEQ ID NO: 3, a CDR2 sequence of SEQ ID NO: 9, and a CDR3 sequence of SEQ ID NO: 16, and a CL domain. In certain preferred embodiments, this binding compound comprises an Fc region that is a human IgG1 Fc region, a human IgG4 Fc region, a silenced human IgG1 Fc region, or a silenced human IgG4 Fc region.

In another preferred embodiment, a bispecific binding compound having binding affinity to a first CD38 epitope and a second, non-overlapping CD38 epitope, the bispecific binding compound comprises: a first polypeptide subunit comprising a heavy chain variable region comprising a CDR1 sequence of SEQ ID NO: 1, a CDR2 sequence of SEQ ID NO: 6, and a CDR3 sequence of SEQ ID NO: 13 in a human heavy chain framework; a second polypeptide subunit comprising a light chain variable region comprising a CDR1 sequence of SEQ ID NO: 49, a CDR2 sequence of SEQ ID NO: 50, and a CDR3 sequence of SEQ ID NO: 51, in a human light chain framework; wherein the first polypeptide subunit and the second polypeptide subunit together have binding affinity to the first CD38 epitope; and a third polypeptide subunit comprising an antigen-binding domain of a heavy-chain antibody comprising a CDR1 sequence of SEQ ID NO: 3, a CDR2 sequence of SEQ ID NO: 9, and a CDR3 sequence of SEQ ID NO: 16 in a human heavy chain framework, in a monovalent or bivalent configuration; wherein the third polypeptide subunit has binding affinity to the second, non-overlapping CD38 epitope. In some preferred embodiments, the first polypeptide subunit further comprises a CH1 domain, at least a portion of a hinge region, a CH2 domain, and a CH3 domain. In some preferred embodiments, the third polypeptide subunit further comprises a constant region sequence comprising at least a portion of a hinge region, a CH2 domain, and a CH3 domain, in the absence of a CH1 domain. In some preferred embodiments, the human light chain framework is a human kappa light chain framework or a human lambda light chain framework. In some preferred embodiments, the second polypeptide subunit further comprises a CL domain. In some preferred embodiments, the bispecific binding compound comprises an Fc region selected from the group consisting of: a human IgG1 Fc region, a human IgG4 Fc region, a silenced human IgG1 Fc region, and a silenced human IgG4 Fc region. In some preferred embodiments, the bispecific binding compound comprises an asymmetric interface between the CH3 domain of the first polypeptide subunit and the CH3 domain of the third polypeptide subunit.

Aspects of the invention include combinations (e.g., therapeutic combinations) of two or more binding compounds described herein. In some embodiments, a therapeutic combination comprises a first binding compound that has binding specificity for a first epitope on CD38, and a second binding compound that has binding specificity for a second, non-overlapping epitope on CD38. Therapeutic combinations in accordance with embodiments of the invention can comprise two or more of the binding compound described herein, or can comprise one or more of the binding compounds described herein, as well as one or more binding compounds known in the art, e.g., one or more second antibodies that bind to CD38.

For example, isatuximab (SAR650984), which is an antibody in clinical trials for the treatment of Multiple Myeloma, induces potent complement dependent cytotoxicity (CDC), antibody dependent cell-mediated cytotoxicity (ADCC), antibody dependent cellular phagocytosis (ADCP), and indirect apoptosis of tumor cells. Isatuximab also blocks the cyclase and hydrolase enzymatic activities of CD38 and induces direct apoptosis of tumor cells. Aspects of the invention include therapeutic combinations that include one or more of the binding compounds described herein, as well as isatuximab. The heavy chain variable region sequence of isatuximab is provided in SEQ ID NO: 30, and the light chain variable region sequence of isatuximab is provided in SEQ ID NO: 31. Isatuximab is described, for example, in Deckert, J., et al., “SAR650984, a novel humanized CD38-targeting antibody, demonstrates potent antitumor activity in models of multiple myeloma and other CD38+hematologic malignancies.” Clin Cancer Res, 2014. 20(17): p. 4574-83, the disclosure of which is incorporated herein by reference in its entirety.

Daratumumab, an antibody specific for human CD38, was approved for human use in 2015 for the treatment of Multiple Myeloma (reviewed in Shallis et al., Cancer Immunol. Immunother., 2017, 66(6):697-703). Aspects of the invention include therapeutic combinations that include one or more of the binding compounds described herein, as well as daratumumab.

In one preferred embodiment, a therapeutic combination comprises a heavy-chain antibody that binds to CD38, the heavy-chain antibody comprising an antigen-binding domain comprising a CDR1 sequence of SEQ ID NO: 4, a CDR2 sequence of SEQ ID NO: 11, and a CDR3 sequence of SEQ ID NO: 17, and isatuximab as a second antibody that binds to CD38.

### Preparation of Anti-Ectoenzyme Binding Compounds

The binding compounds of the present invention can be prepared by methods known in the art.

In a preferred embodiment, the binding compounds herein are produced by transgenic animals, including transgenic mice and rats, preferably rats, in which the endogenous immunoglobulin genes are knocked out or disabled. In a preferred embodiment, the binding compounds herein are produced in UniRat™. UniRat™ have their endogenous immunoglobulin genes silenced and use a human immunoglobulin heavy-chain translocus to express a diverse, naturally optimized repertoire of fully human heavy-chain antibodies. While endogenous immunoglobulin loci in rats can be knocked out or silenced using a variety technologies, in UniRat™ the zinc-finger (endo)nuclease (ZNF) technology was used to inactivate the endogenous rat heavy chain J-locus, light chain Cκ locus and light chain Cλ locus. ZNF constructs for microinjection into oocytes can produce IgH and IgL knock out (KO) lines. For details see, e.g., Geurts et al., 2009, Science 325:433. Characterization of Ig heavy chain knockout rats has been reported by Menoret et al., 2010, Eur. J. Immunol. 40:2932-2941. Advantages of the ZNF technology are that non-homologous end joining to silence a gene or locus via deletions up to several kb can also provide a target site for homologous integration (Cui et al., 2011, Nat Biotechnol 29:64-67). Human heavy-chain antibodies produced in UniRat™ are called UniAbs™ and can bind epitopes that cannot be attacked with conventional antibodies. Their high specificity, affinity, and small size make them ideal for mono- and poly-specific applications.

In addition to UniAbs™, specifically included herein are heavy chain-only antibodies lacking the camelid VHH framework and mutations, and their functional VH regions. Such heavy chain-only antibodies can, for example, be produced in transgenic rats or mice which comprise fully human heavy chain-only gene loci as described, e.g., in WO2006/008548, but other transgenic mammals, such as rabbit, guinea pig, rat can also be used, rats and mice being preferred. Heavy chain-only antibodies, including their VHH or VH functional fragments, can also be produced by recombinant DNA technology, by expression of the encoding nucleic acid(s) in a suitable eukaryotic or prokaryotic host, including, for example, mammalian cells (e.g., CHO cells), E. coli or yeast.

Domains of heavy chain-only antibodies combine advantages of antibodies and small molecule drugs: can be mono- or multi-valent; have low toxicity; and are cost-effective to manufacture. Due to their small size, these domains are easy to administer, including oral or topical administration, are characterized by high stability, including gastrointestinal stability; and their half-life can be tailored to the desired use or indication. In addition, VH and VHH domains of heavy-chain antibodies can be manufactured in a cost-effective manner.

In a particular embodiment, the heavy chain antibodies of the present invention, including UniAbs™, have the native amino acid residue at the first position of the FR4 region (amino acid position 101 according to the Kabat numbering system), substituted by another amino acid residue, which is capable of disrupting a surface-exposed hydrophobic patch comprising or associated with the native amino acid residue at that position. Such hydrophobic patches are normally buried in the interface with the antibody light chain constant region but become surface exposed in heavy-chain antibodies and are, at least partially, responsible for the unwanted aggregation and light chain association of heavy-chain antibodies. The substituted amino acid residue preferably is charged, and more preferably is positively charged, such as lysine (Lys, K), arginine (Arg, R) or histidine (His, H), preferably arginine (R). In a preferred embodiment, the heavy chain-only antibodies derived from the transgenic animals contain a Trp to Arg mutation at position 101. The resultant heavy-chain antibodies preferably have high antigen-binding affinity and solubility under physiological conditions in the absence of aggregation.

In certain embodiments, a binding compound is an anti-ectoenzyme heavy chain antibody that binds to CD38. In a preferred embodiment, the anti-CD38 heavy chain antibodies are UniAbs™.

As part of the present invention, human IgG heavy chain anti-CD38 antibody families with unique CDR3 sequences from UniRat™ animals (UniAb™) were identified that bind human CD38 in ELISA (recombinant CD38 extracellular domain) protein and cell-binding assays. Heavy chain variable region (VH) sequences comprising three sequence families (F11, F12 and F13, see FIGS. 1-3 and 5) are positive for human CD38 protein binding and/or for binding to CD38+ cells, and are all negative for binding to cells that do not express CD38. UniAbs™ from these three sequence families fall into two broad synergistic groups based on the ability to inhibit the hydrolase function of CD38.

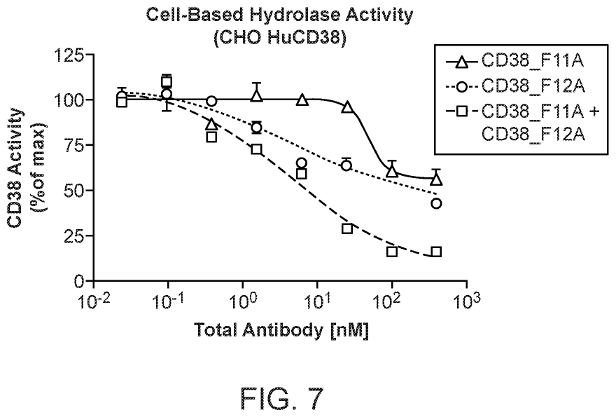

One synergistic group includes the F11 and F12 sequence families. The members of the F11/F12 synergistic group do not synergize with Isatuximab to inhibit the hydrolase function of CD38, but do exhibit synergistic hydrolase inhibition with one another. For example, when combined, F11A and F12A achieve a hydrolase inhibition level that is greater than either F11A or F12A can achieve individually (FIG. 7).

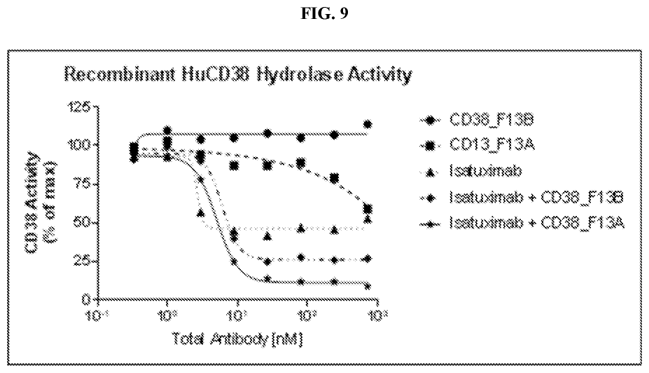

Another synergistic group includes the F13 sequence family and Isatuximab. Isatuximab alone elicits a partial inhibition of the hydrolase activity of CD38 (˜55% inhibition, FIG. 9). F13A alone also elicits partial inhibition of the hydrolase activity of CD38. When combined, Isatuximab and F13A demonstrate synergistic inhibition of hydrolase activity by achieving a reduction in hydrolase activity that is greater than that achieved by either antibody individually. Some members of the F13 synergistic group do not block CD38 hydrolase activity on their own, but synergize with Isatuximab to do so. For example, F13B does not block CD38 hydrolase activity by itself, but synergizes with Isatuximab to inhibit CD38 hydrolase activity by up to 75% (e.g., FIG. 9).

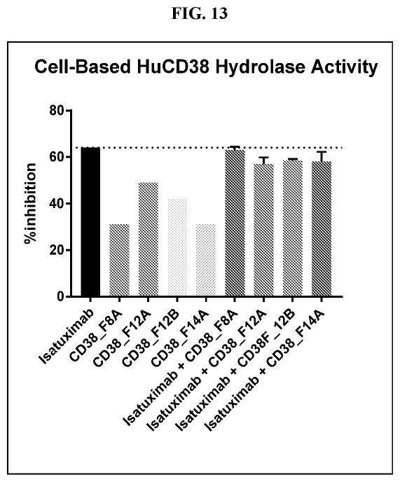

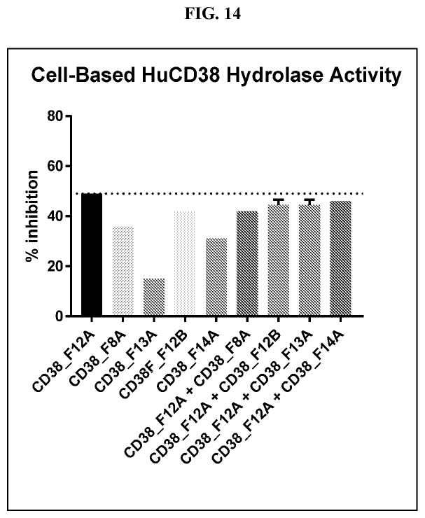

Notably, F12A inhibits CD38 hydrolase activity by itself (˜50% inhibition, FIGS. 13-14), but does not synergize with Isatuximab. The combination of F12A and Isatuximab resulted in slightly lower inhibition than Isatuximab alone (˜65% for Isatuximab alone versus ˜58% for the combination of Isatuximab and F12A).

Combinations of two or more UniAbs™ binding to distinct, non-overlapping epitopes induce potent CDC activity and direct apoptosis, whereas the same UniAbs™, when administered alone, do not induce either of these effector functions. Combinations of UniAbs™ also inhibited enzymatic activities more potently than the individual UniAbs™ when administered alone. In other words, in certain embodiments, a combination of two different binding compounds (e.g., a therapeutic combination) of the present invention results in one or more synergistic results (e.g., synergistic CDC activity, synergistic enzymatic modulation activity, e.g., synergistic hydrolase blocking activity).

Binding compounds in accordance with embodiments of the invention bind to CD38-positive Burkitt's lymphoma cell line Ramos, and are cross-reactive with the CD38 protein of Cynomolgus macaque. In addition, they can be engineered to provide cross-reactivity with the CD38 protein of any animal species, if desired.

Binding compounds in accordance with embodiments of the invention may have an affinity for CD38 with a Kd of from from about 10⁻⁶ to around about 10⁻¹¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating, e.g. blocking, a CD38 biological activity, including in vitro assays, pre-clinical models, and clinical trials, as well as assessment of potential toxicity.

Binding compounds in accordance with embodiments of the invention which bind to two or more non-overlapping epitopes on an ectoenzyme target, including but not limited to anti-CD38 heavy chain antibodies, e.g. UniAbs™ can be identified by competition binding assays, such as enzyme-linked immunoassays (ELISA assays) or flow cytometric competitive binding assays. For example, one can use competition between known antibodies binding to the target antigen and the antibody of interest. By using this approach, one can divide a set of antibodies into those that compete with the reference antibody and those that do not. The non-competing antibodies are identified as binding to a distinct epitope that does not overlap with the epitope bound by the reference antibody. Often, one antibody is immobilized, the antigen is bound, and a second, labeled (e.g., biotinylated) antibody is tested in an ELISA assay for ability to bind the captured antigen. This can be performed also by using surface plasmon resonance (SPR) platforms, including ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc). For further details see the Examples section below.

Typically, a binding compound (e.g., an antibody) competes with a reference binding compound (e.g., a reference antibody) if it causes about 15-100% reduction in the binding of the reference antibody to the target antigen, as determined by standard techniques, such as by the competition binding assays described herein. In various embodiments, the relative inhibition is at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95% or higher.

### Pharmaceutical Compositions

It is another aspect of the present invention to provide pharmaceutical compositions comprising one or more binding compounds of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

In one embodiment, a pharmaceutical composition comprises two or more heavy-chain antibodies binding to non-overlapping epitopes on an ectoenzyme, such as, for example, CD38, CD73, or CD39. In a preferred embodiment, the pharmaceutical compositions comprise synergistic combinations of two or more heavy-chain antibodies binding to non-overlapping epitopes of an ectoenzyme, such a, for example, CD38, CD73, or CD39.

In another embodiment, a pharmaceutical composition comprises a multi-specific (including bispecific) heavy-chain antibody with binding specificity for two or more non-overlapping epitopes on an ectoenzyme, such as, for example, CD38, CD73, or CD39. In a preferred embodiment, a pharmaceutical composition comprises a multi-specific (including bispecific) heavy-chain antibody with binding specificity to two or more non-overlapping epitopes on an ectoenzyme, e.g., CD38, CD73, or CD39, having synergistically improved properties relative to any of the monospecific antibodies binding to the same epitope.

Pharmaceutical composition of the binding compounds used in accordance with the present invention are prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The binding compounds herein can be administered by intravenous injection or infusion or subcutaneously. For injection administration, the binding compounds herein can be formulated in aqueous solutions, preferably in physiologically-compatible buffers to reduce discomfort at the site of injection. The solution can contain carriers, excipients, or stabilizers as discussed above. Alternatively, binding compounds can be in lyophilized form for constitution with a suitable vehicle, e.g., sterile pyrogen-free water, before use.

Anti-CD38 antibody formulations are disclosed, for example, in U.S. Pat. No. 9,034,324. Similar formulations can be used for the heavy chain antibodies, including UniAbs™, of the present invention. Subcutaneous antibody formulations are described, for example, in US 20160355591 and US 20160166689.

### Articles of Manufacture

Aspects of the invention include articles of manufacture, or “kits”, containing one or more binding compounds of the invention that are useful for the treatment of the diseases and disorders described herein. In one embodiment, a kit comprises a container comprising an anti-CD38 binding compound as described herein. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister packs, etc. The container may be formed from a variety of materials such as glass or plastic. The container may hold one or more anti-CD38 binding compounds as described herein, or a formulation thereof, e.g., a combination formulation of two or more anti-CD38 binding compounds, which is effective for treating a condition and may have a sterile access port (for example, the container may be an intravenous solution bag or a vial having a stopper pierceable by a hypodermic injection needle). The label or package insert indicates that the composition is used for treating the condition of choice, such as a cancer or an immunological disorder. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of one or more binding compounds and, if present, a combination formulation thereof. For example, if the kit comprises a first pharmaceutical composition comprising a first anti-CD38 binding compound and a second pharmaceutical composition comprising a second anti-CD38 binding compound, the kit may further comprise directions for the simultaneous, sequential or separate administration of the first and second pharmaceutical compositions to a patient in need thereof Where a kit comprises two or more compositions, the kit may comprise a container for containing the separate compositions, such as a divided bottle or a divided foil packet, however, the separate compositions may also be contained within a single, undivided container. A kit can comprise directions for the administration of the separate components, or for the administration a combined formulation thereof.

### Methods of Use

The binding compounds described herein, which bind to non-overlapping epitopes on an ectoenzyme, combinations, including synergistic combinations, of such binding compounds, multi-specific antibodies with binding specificities to two or more non-overlapping epitopes on an ectoenzyme, and pharmaceutical compositions comprising such antibodies and antibody combinations, can be used to target diseases and conditions characterized by the expression of the target ectoenzyme.

In various embodiments, the ectoenzyme is selected from the group consisting of CD10, CD13, CD26, CD38, CD39, CD73, CD156b, CD156c, CD157, CD203, VAP1, ART2, and MT1-MMP.

In a particular embodiment, the ectoenzyme is CD38, CD73 and/or CD39.

CD38 is a 46-kDa type II transmembrane glycoprotein with a short 20-aa N-terminal cytoplasmic tail and a long 256-aa extracellular domain (Malavasi et al., Immunol. Today, 1994, 15:95-97). Due to its high level of expression in a number of hematological malignancies, including multiple myeloma (MM), non-Hodgkin's lymphoma (reviewed in Shallis et al., Cancer Immunol. Immunother., 2017, 66(6):697-703), B-cell chronic lymphocylic leukemia (CLL) (Vaisitti et al., Leukemia, 2015, 29″356-368), B-cell acute lymphoblastic leukemia (ALL), an dT-cell ALL, CD38 is a promising target for antibody-based therapeutics to treat hematological malignancies. CD38 has also be implicated as a key actor in age-related nicotinamide adenine dinucleotide (NAD) decline, and it has been suggested that CD38 inhibition, combined with NAD precursors may serve as a potential therapy for metabolic dysfunction and age-related diseases (see, e.g., Camacho-Pereira et al., Cell Metabolism 2016, 23:1127-1139). CD38 has also been described as being involved in the development of airway hyper-responsiveness, a hallmark feature of asthma, and has been suggested as a target to treat such conditions.

Nicotinamide adenine dinucleotide (NAD+) metabolism plays a critical role in many inflammatory disorders, including metabolic diseases and Alzheimer's disease. NAD is a major coenzyme in bioenergetic processes and its cleavage by several enzymes, including CD38, is key to many biological processes such as cell metabolism, inflammatory responses and cell death (Chini et al., Trends Pharmacol Sci, 39(4):424-36.

The NAD cleaving enzyme, CD38, promotes intestinal inflammation in animal models. CD38 is a multifunctional ectoenzyme involved in the degradation of NAD+ and the production of cell-activating metabolites such as adenosine diphosphate ribose (ADPR) and cyclic ADPR (cADPR). CD38 is mainly expressed on hematopoietic cells, such as T cells, B cells, and macrophages Immune cells upregulate expression of CD38 after activation and differentiation. Based on animal studies, it appears that immune responses of both T cells, macrophages and neutrophils are modulated by CD38. High-level CD38 expression and its associated ectoenzymatic functions seem to enhance the development of inflammatory diseases. In contrast, CD38 deficiency, and concomitant increased NAD concentrations, reduces recruitment of cells to inflamed sites and reduces production of pro-inflammatory cytokines (Schneider et al., PLos One, 10(5): e0126007 (2015); Gerner et al., Gut, 6 Sep. 2017, doi: 10.1136/gutjnl-2017-314241; Garcia-Rodriguez et al., Sci Rep, 8(1): 3357 (2018)). In autoimmune models, CD38−/− mice show ameliorated development of disease, less joint inflammation in a collagen-induced arthritis model and less inflammation of the gut in a dextran sulfate sodium (DSS) colitis model (Garcia-Rodriguez et al., Sci Rep, 8(1): 3357 (2018)). All these results combined support the hypothesis that colonic inflammation leads to a decrease in NAD levels in cells via activation of CD38. The subsequent NAD decline would decrease the activity of the NAD-dependent deacetylases (sirtuins) that are known to have anti-inflammatory and tissue protective effects.

Monoclonal antibodies against CD38 have been shown to be highly efficacious in the treatment of Multiple Myeloma (MM), however, they are not suitable for the treatment of IBD. Currently, four monoclonal antibodies are in clinical trials for the treatment of CD38+ malignancies. The most advanced is Daratumumab (Janssen Biotech) which was approved for human use by the FDA for the treatment of MM in 2015. All three anti-CD38 monoclonals antibodies in clinical trials for MM show similar favorable safety and efficacy profiles (van de Donk, et al., Blood 2017, blood-2017-06-740944; doi: https://doi.org/10.1182/blood-2017-06-740944). One monoclonal antibody (TAK-079) is in clinical trials for the treatment of auto-immune diseases including Systemic Lupus Erythematosis (SLE) and rheumatoid arthritis. Besides plasma cells, anti-CD38 monoclonal antibodies deplete other CD38+ cells in the spleen and blood, including all NK cells and ˜50% of monocytes, T cells and B cells. Critical regulatory immune cells, such as Treg cells and Myeloid Derived Suppressor Cells (MDSC), are depleted in MM patients after treatment with anti-CD38 monoclonal antibodies, and expansion of effector T cells is observed (Krejcik, et al., Blood, 128(3): 384-94 (2016)). In all likelihood, expansion of anti-tumor effector T cells contributes to the effectiveness of anti-CD38 mAbs in MM. However, removing important regulatory immune cells in auto-immune diseases could lead to exacerbation of disease.

Inhibition of enzyme function of CD38 could be a safe and effective approach to treating inflammatory disorders. Several small molecule inhibitors, including one with strong potency (Kd-5 nM, Haffner et al 2015) of CD38 have been developed (Haffner et al., J Med. Chem, 58(8): 3548-71 (2015)). This compound elevated NAD levels in tissues of mice 6 hours post-injection, indicating that inhibition of CD38 leads to higher intracellular NAD in mice. However, CD38 is also expressed in the brain and plays a role in behavior, so that such molecules have significant risk of toxicity. In contrast to small molecule compounds, antibodies cannot cross the blood-brain barrier, and generally have superior target specificity compared to small molecules and thus should have a significantly better safety profile. Inflammatory diseases include Multiple Sclerosis, Systemic Lupus Erythematosus, rheumatoid arthritis, Graft versus Host disease, etc.

Antibodies in clinical trials were selected on the basis of cytolysis and poorly inhibit biological functions of CD38, but modulation of these functions may also be relevant for cancer therapies. Recent papers by Chatterjee et al. and Chen et al. established that the CD38-NAD+ axis is important in preclinical models of lung cancer and melanoma. These studies indicate that high levels of NAD+, negatively regulated by CD38, preserve effector T cell (Teff) functionality.

The binding compounds described herein, including heavy chain only anti-CD38 antibodies, antibody combinations, multi-specific antibodies, and pharmaceutical compositions herein can be used to target diseases and conditions characterized by the expression or overexpression of CD38, including, without limitation, the conditions and diseases listed above.

In one aspect, the CD38 binding compounds and pharmaceutical compositions herein can be used to treat hematological malignancies characterized by the expression of CD38, including multiple myeloma (MM), non-Hodgkin's lymphoma, B-cell chronic lymphocylic leukemia (CLL), B-cell acute lymphoblastic leukemia (ALL), and T-cell ALL. The CD38 binding compounds and pharmaceutical compositions of the present invention can also be used to treat asthma and other conditions characterized by airway hyper-responsiveness, and age-related, and metabolic dysfunction characterized by micorinamide adenine dinucleotide (NAD) decline. The CD38 binding compounds and pharmaceutical compositions of the present invention can also be used to treat colitis.

MM is a B-cell malignancy characterized by a monoclonal expansion and accumulation of abnormal plasma cells in the bone marrow compartment. Current therapies for MM often cause remissions, but nearly all patients eventually relapse and die. There is substantial evidence of an immune-mediated elimination of myeloma cells in the setting of allogeneic hematopoietic stem cell transplantation; however, the toxicity of this approach is high, and few patients are cured. Although some monoclonal antibodies have shown promise for treating MM in preclinical studies and early clinical trials, consistent clinical efficacy of any monoclonal antibody therapy for MM has not been conclusively demonstrated. There is therefore a great need for new therapies, including immunotherapies for MM (see, e.g. Shallis et al, supra).

The CD38 binding compounds and pharmaceutical compositions herein can be also be used to treat autoimmune disorders, including, but not limited to, rheumatoid arthritis (RA), pemphigus vulgaris (PV), systemic lupus erythematosus (SLE), systemic sclerosis (systemic scleroderma, diffuse scleroderma), fibrosis, and multiple sclerosis (MS). The CD38 binding compounds and pharmaceutical compositions herein can be also be used to treat ischemic injuries, including, but not limited to, ischemic brain injuries, ischemic cardiac injuries, ischemic GI injuries, and ischemic kidney injuries (e.g., acute kidney ischemic injuries).

CD73 has been described to function as an ectoenzyme to produce extracellular adenosine, which promotes tumor growth by limiting antitumor T-cell immunity via adenosine receptor signaling. CD73 is expressed in certain cancers, such as breast, colon and prostate cancers. Results with small molecule inhibitors or monoclonal antibodies targeting CD73 in murine tumor models suggest the potential of targeted CD73 therapy, including immunotherapy, to control growth of tumors characterized by the expression of CD73, as monotherapy or in combination with other anticancer agents.

CD39 and CD73 have been widely considered pivotal in the generation of immunosuppressive microenvironments through adenosine production. Upregulation of CD39 has been reported in a number of epithelial and hematological malignancies and its expression in chronic lymphocytic leukemia has been shown to correlate with poor prognosis (Pulte et al., 2011, Clin Lymphoma Myeloma Leuk. 2011; 11:367-372; Bastid et al., 2013, Oncogene, 32:1743-1751; Bastid et al., 2015, Cancer Immunol Res., 3:254-265). CD39 is also highly expressed on regulatory T-cells (Tregs) and is required for their suppressive function as demonstrated with impaired suppressive activity of Tregs in CD39-null mice (Deaglio et al., 2007, J Exp Med., 204:1257-1265). It has been suggested that CD39 may help drive tumorigenesis by its enhanced enzymatic activity either on Tregs, tumor-associated stroma or on malignant epithelial cells, resulting in adenosine-mediated immunosuppression of anti-tumor T- and natural killer (NK) cells as well as neutralization of ATP-induced cell death by chemotherapy (Bastid et al., 2013 and 2015, supra; Feng et al., 2011, Neoplasia, 13:206-216). Modulation of the immunosuppressive CD39/CD73-adenosine pathway has been suggested as a promising immunotherapeutic strategy for cancer therapy (Sitkovsky et al., 2014, Cancer Immunol Res. 2:598-605). See also, Hayes et al., Am J Trans Res, 2015, 7(6):1181-1188.

For a review of the role of CD73 and CD39 ectonucleotidases in T cell differentiation, see, e.g., Bono et al., FEBS Letters, 2015, 589:3454-3460.

Effective doses of the compositions of the present invention for the treatment of disease vary depending upon many different factors, including means of administration, target site, physiological state of the patient, whether the patient is a human or another animal, other medications administered, and whether treatment is prophylactic or therapeutic. Usually, the patient is a human, but non-human mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration. Dosage unit forms generally contain between from about 1 mg to about 500 mg of an active ingredient.

In some embodiments, the therapeutic dosage the agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Typically, compositions are prepared as injectables, either as liquid solutions or suspensions; solid forms suitable for solution in, or suspension in, liquid vehicles prior to injection can also be prepared. The pharmaceutical compositions herein are suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the binding compounds described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the binding compounds described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise a binding compound of the invention dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are articles of manufacture, or “kits” (as described above) comprising the active agents and formulations thereof, of the invention and instructions for use. The kit can further contain a least one additional reagent, e.g. a chemotherapeutic drug, etc. Kits typically include a label indicating the intended use of the contents of the kit. The term “label” includes any writing, or recorded material supplied on or with the kit, or which otherwise accompanies the kit.

The invention now being fully described, it will be apparent to one of ordinary skill in the art that various changes and modifications can be made without departing from the spirit or scope of the invention.

### Materials and Methods

The following materials and methods were utilized to carry out the examples described below.

### CD38 Cell Binding

Binding to CD38 positive cells was assessed by flow cytometry (Guava easyCyte 8HT, EMD Millipore) using the Ramos cell line (ATCC) or CHO cells stably expressing human CD38. Briefly, 100,000 target cells were stained with a dilution series of purified UniAbs™ for 30 minutes at 4° C. Following incubation, the cells were washed twice with flow cytometry buffer (1×PBS, 1% BSA, 0.1% NaN₃) and stained with goat F(ab′)₂ anti-human IgG conjugated to R-phycoerythrin (PE) (Southern Biotech, cat. #2042-09) to detect cell-bound antibodies. After a 20-minute incubation at 4° C., the cells were washed twice with flow cytometry buffer and then mean fluorescence intensity (MFI) was measured by flow cytometry.

### Antibody-Induced Direct Apoptosis

Cytotoxicity through antibody-induced direct apoptosis was analyzed using CD38 positive Ramos cells (ATCC). In summary, 45,000 target cells were treated with 2 μg/mL of purified UniAbs™ for 48 hours (37° C., 8% CO₂). Following incubation, the cells were washed twice with Annexin-V binding buffer (BioLegend, cat. #422201) and stained with Annexin V and 7-AAD (BioLegend, cat. #640945 and 420404). The samples were then analyzed by flow cytometry (Guava easyCyte 8HT, EMD Millipore) and the percentage of viable cells was determined as the population negative for Annexin V and 7AAD.

### CD38 Hydrolase Activity Assay

To measure inhibition of CD38 hydrolase activity, recombinant human CD38 protein (Sino Biological) or human CD38-expressing CHO cells (125,000 cells/well) were incubated with each purified anti-CD38 UniAb™ in hydrolase activity buffer (40 mM Tris, 250 mM Sucrose, 25 μg/mL BSA, pH 7.5) for 15 minutes at room temperature. After incubation, ε-NAD⁺ (BioLog Cat. No. N010) was added to a final concentration of 150 μM. Production of a fluorescent product was measured at 1 hour (ex 300 nm/em 410 nm) using a Spectramax i3x plate reader (Molecular Devices). Hydrolase enzyme inhibition was assessed by comparing signal from UniAb™-treated wells to the percent of total enzymatic activity observed when CD38 protein was treated with an isotype control antibody (max).

### EXAMPLES

### Example 1

### Gene Assembly, Expression and Sequencing

cDNAs encoding heavy-chain only antibodies highly expressed in lymph node cells were selected for gene assembly and cloned into an expression vector. Subsequently, these heavy chain sequences were expressed in HEK cells as UniAb™ heavy chain only antibodies (CH1 deleted, no light chain).

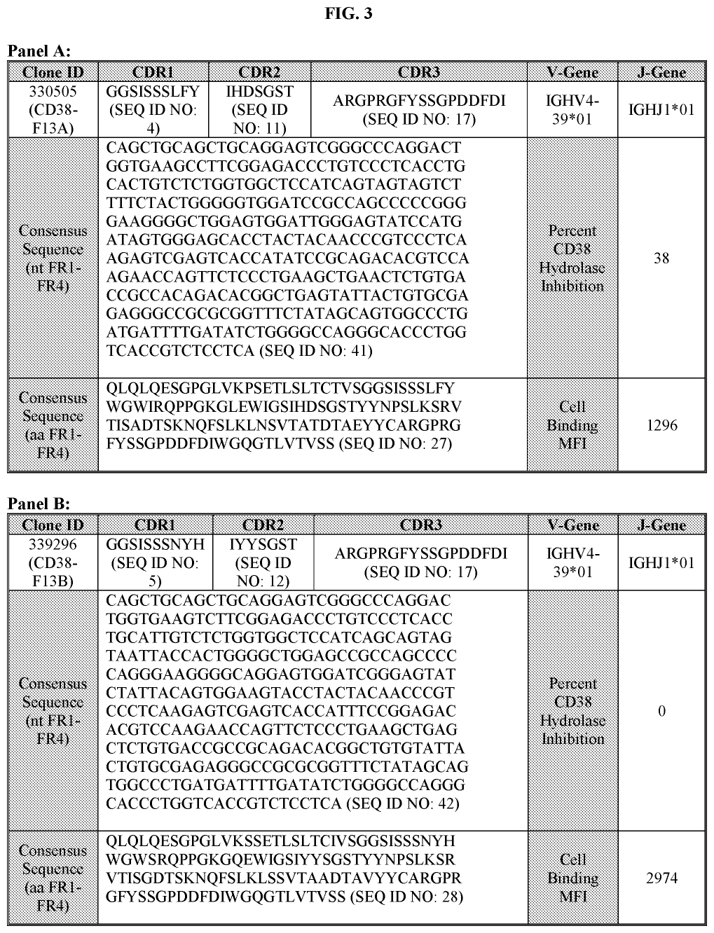

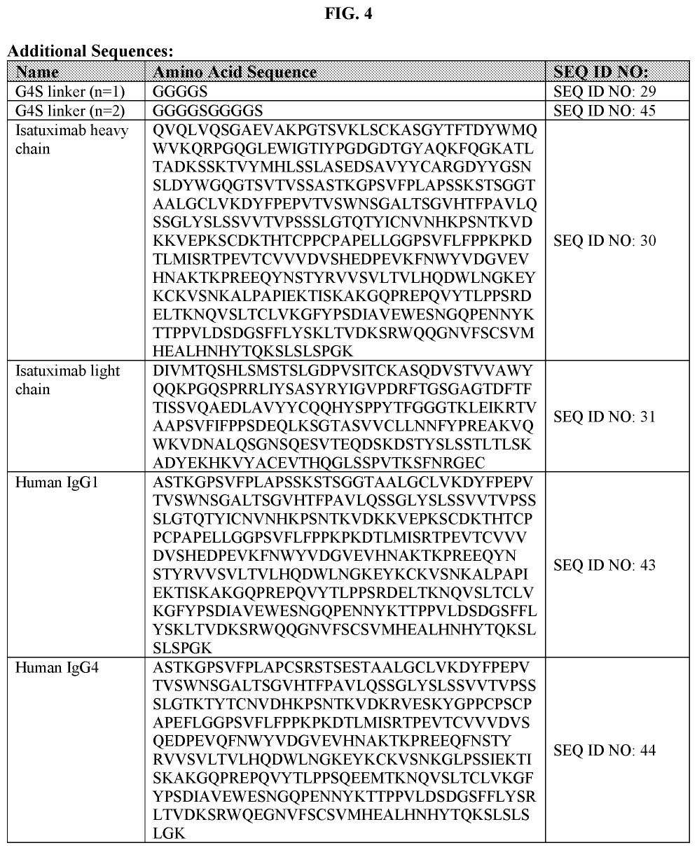

FIGS. 1, 2, 3 and 5 show the heavy chain variable domain amino acid sequences of anti-CD38 UniAb™ families CD38_F11, CD38_F12 and CD38_F13, respectively. These figures indicate the clone ID of the UniAb™ tested, the percentage inhibition of hydrolase enzymatic activity of recombinant CD38 in the presence of the respective CD38-binding UniAbs™ versus control UniAb™, and the mean fluorescent intensity (MFI) of cell binding to Ramos cells. Also provided in FIGS. 1, 2, 3 and 5 are the sequences (CDR sequences, variable region sequences, (both amino acid and nucleotide)), as well as the VH and VJ gene usage of CD38 binding heavy chain antibodies of families F11, F12 and F13, respectively. Additional sequences are provided in FIG. 4.

### Example 2

### Cell Binding of Anti-CD38 UniAbs

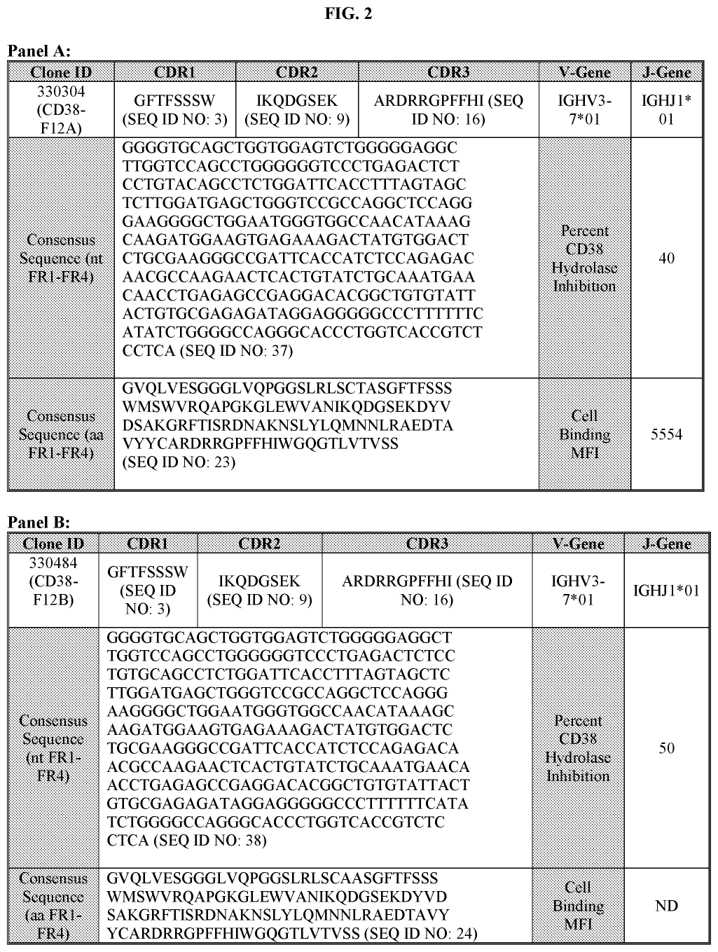

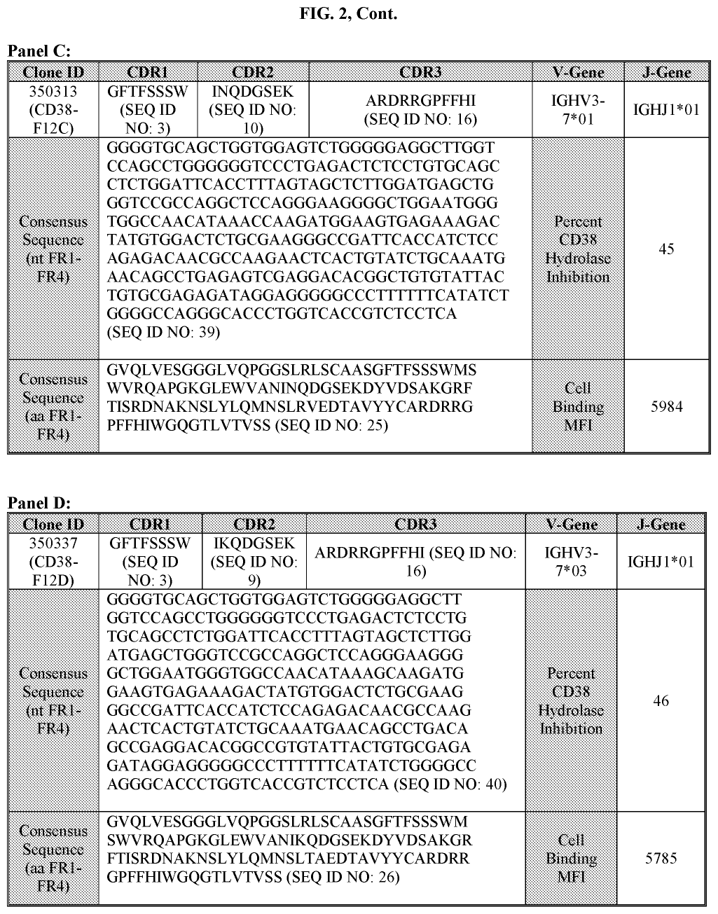

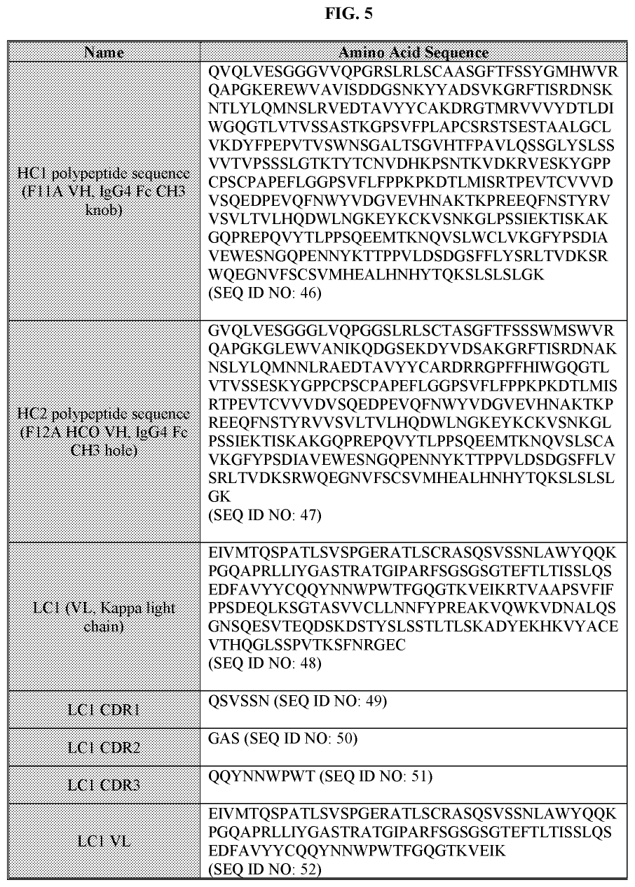

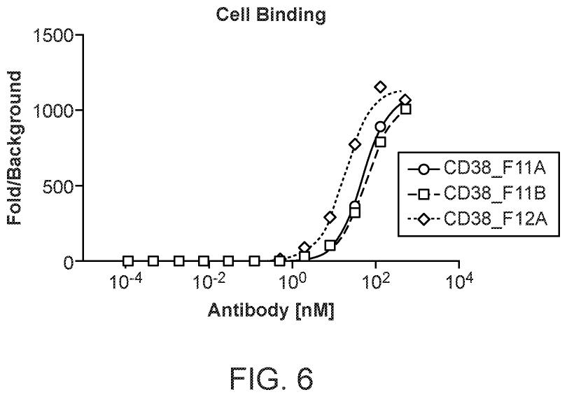

FIGS. 1-2 provide cell binding data for binding to Ramos cells for CD38_F11 and CD38_F12 family members. FIG. 6 shows binding of anti-CD38 UniAb™ CD38_F11 and CD38_F12 antibodies at different concentrations to CHO cells stably transfected with human CD38.

### Example 3

### Synergies of CD38 Binding Heavy Chain Antibodies in Blocking Hydrolase Activity of CD38

As shown in FIG. 7, UniAbs™ representing two unique heavy chain CDR3 sequence families, CD38_F11 and CD38_F12, partially inhibited the hydrolase activity of CD38 when administered alone, but when mixed (i.e., combined) at equimolar concentrations, inhibited CD38 hydrolase activity more strongly.

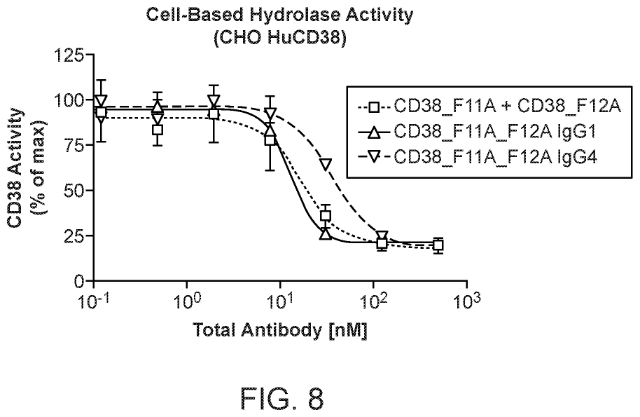

FIG. 8 shows enzyme inhibition of the hydrolase activity of CD38 by bivalent UniAbs™. A mixture of two anti-CD38 UniAbs™ (CD38_F11A+CD38_F12A) was equally effective as a bivalent heavy chain antibody with one arm with the VH of CD38_F11A and the other arm with the VH of CD38_F12A (CD38_F11A_F12A) in inhibiting hydrolase activity on cells. Biparatopic UniAbs™ (CD38_F11A_F12A) having an IgG1 Fc tail or an IgG4 Fc tail both inhibited hydrolase activity on cells. These UniAbs™ and their VH domains bind to two non-overlapping epitopes on CD38.

FIG. 9 shows enzyme inhibition of the hydrolase activity of CD38 by a mixture of either UniAbs™ CD38_F13A or CD38_F13B with Isatuximab. Isatuximab alone partially inhibited CD38 hydrolase activity, but combinations of Isatuximab with CD38_F13A or CD38_F13B inhibited enzyme activity more strongly, demonstrating a synergistic effect.

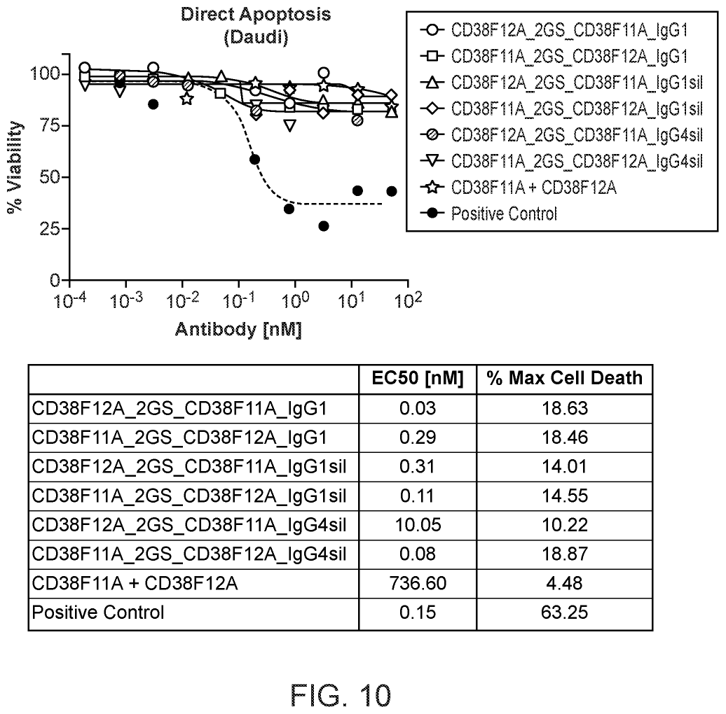

FIG. 10 shows direct cytotoxicity of Daudi cells. UniAb™ CD38_F11A was mixed with an equimolar amount of CD38_F12A and shown not to induce apoptosis of Daudi cells. Biparatopic, bivalent antibodies comprising the VHs of CD38_F11A and CD38_F12A also did not kill Daudi cells. Isatuximab was used as a positive control and shown to potently kill Daudi cells.

FIG. 11 shows a schematic representation of two bivalent (Panels C and D) and two tetravalent (Panels A and B) UniAb™ formats in accordance with embodiments of the invention. These schematic representations are non-limiting.

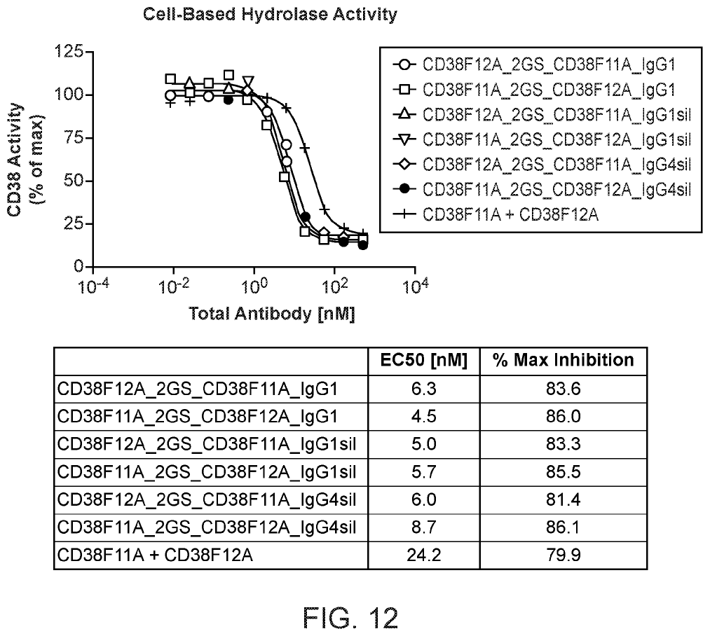

FIG. 12 shows enzyme inhibition of the hydrolase activity of human CD38 expressed on CHO cells by tetravalent UniAbs™ as described in FIG. 11 (Panel B represents the format in this example). The overall design is first the ID of the most distal VH, then the linker Glycine-Glycine-Glycine-Glycine-Serine (GGGGS (SEQ ID NO: 29)) and next the ID of the VH proximal to the Fc tail. Tetravalent UniAbs™ were expressed with human IgG1, silenced human IgG4, and silenced human IgG1. All tetravalent antibodies inhibited the hydrolase activity of CD38 completely and more potently than a mixture of two UniAbs of 330204 (also named CD38F12A) and 309157 (also named CD38F11A). Orientation of VH (proximal or distal from Fc) and Fc isotype showed similar potency.

FIG. 13 shows inhibition of mixtures of UniAbs with Isatuximab. UniAbs and Isatuximab were tested individually at 400 nM and as mixtures at 200 nM of each antibody. Isatuximab inhibited the hydrolase activity of CD38 partially (60%). UniAbs were also partial blockers of the hydrolase activity. Mixtures of these partial blockers failed to inhibit the hydrolase activity of CD38 more potently than Isatuximab by itself.

FIG. 14 shows inhibition of hydrolase activity of CD38 by mixtures of UniAbs. UniAb CD38_F12A was tested individually at 400 nM and mixed with other UniAbs at 200 nM of each antibody. CD38_F12A inhibited the hydrolase activity of CD38 partially (˜50%). Other partial inhibitors of CD38 failed to show synergy with CD38_F12A to inhibit the hydrolase activity of CD38. For example, CD38_F13A shows synergy when combined with Isatuximab, but does not enhance inhibition when administered in combination with CD38_F12A.

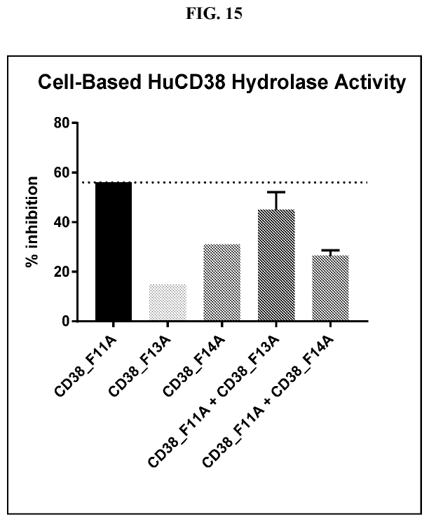

FIG. 15 shows inhibition of hydrolase activity of CD38 by mixtures of UniAbs. UniAb CD38_F11A was tested individually at 400 nM and mixed with other UniAbs at 200 nM of each antibody. CD38_F11A inhibited the hydrolase activity of CD38 partially (˜58%). Other partial inhibitors of CD38 failed to show synergy with CD38_F11A to inhibit the hydrolase activity of CD38. For example, CD38_F13A shows synergy when administered with Isatuximab, but does not enhance inhibition in combination with CD38_F11A.

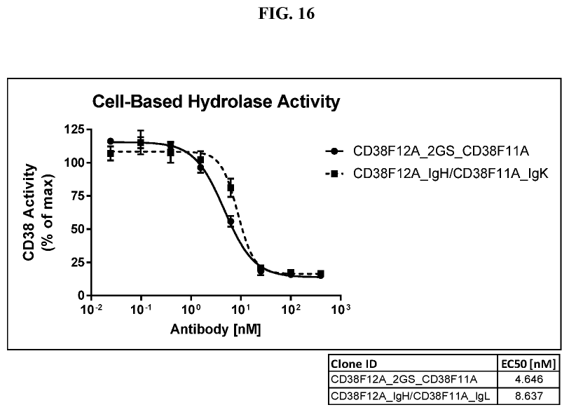

FIG. 16 shows enzyme inhibition of the hydrolase activity of human CD38 expressed on CHO cells by tetravalent UniAbs™ as described in FIG. 11 (Panel B respresents the format of CD38F12A_2GS_CD38F11A, and Panel A represents the format of CD38F12A_IH/CD38F11A_IgK). The overall design is first the antigen-binding domain (ID) of the most distal VH, then the linker Glycine-Glycine-Glycine-Glycine-Serine (GGGGS (SEQ ID NO: 29)) and next the antigen-binding domain (ID) of the VH proximal to the Fc region. Tetravalent UniAbs™ were expressed with a human IgG4 Fc region. All tetravalent antibodies inhibited the hydrolase activity of CD38 completely and with comparable potency (IC50=4.5 nM for Panel B format versus IC50=8.6 nM for Panel A format).

### Example 4

### Efficacy of Hydrolase Inhibitory UniAbs in a DSS Colitis Model

Description of procedures: C57BL/6 mice or human CD38 knock-in mice are given DSS (0.5%-5%) in drinking water. Low doses (0.5%-3%) results in development of chronic colitis and high doses (2%-5%) in development of acute colitis. Colitis will be followed by measuring body weight, occult blood and other markers of inflammation of the gut (Chassaing, B., et al., “Dextran sulfate sodium (DSS)-induced colitis in mice.” Curr Protoc Immunol, 2014. 104: p. Unit 15 25). Body weight, histological examination of intestinal tissues, and colon length is used to assess efficacy of treatment (Chassaing, B., et al., “Dextran sulfate sodium (DSS)-induced colitis in mice”, Curr Protoc Immunol, 2014, 104: p. Unit 15 25). Mice are treated by injecting intravenously selected UniAbs once, twice, or three times per week at doses ranging from 0.5 mg/kg to 5 mg/kg.

Choice of Animal and Species: The experiments are conducted in human CD38 knock-in models or wild-type mice. C57BL/C mouse strains or other susceptible mouse strains are used and are a widely accepted model of IBD in humans. Sex: Males and females; Age: from 4 weeks to 2-3-year-old. Weight: variable.

Generation of a human CD38 constitutive knockin model in C57BL/6 mice: The coding sequence of exon 1 plus partial intron 1 are replaced with a “human CD38 CDS-polyA” cassette. To engineer the targeting vector, homology arms are generated by PCR using BAC clone RP24-163F10 or RP23-58C20 from the C57BL/6 library as template. In the targeting vector, the Neo cassette is flanked by SDA (self-deletion anchor) sites. DTA is used for negative selection. C57BL/6 ES cells are used for gene targeting. Founder animals heterozygous for the human CD38 transgene are produced and, subsequently, are bred to homozygocity.

Sample sizes: 8 or more animals per group are exposed to DSS in drinking water and treated with hydrolase blocking or control antibodies. Some measurements are repeated at least 2-3 times to offer solid biological and statistical power. In general, past biochemical and physiological studies indicated that a sample size of n=4-6 animals provides adequate statistical power (i.e., 80% power) to detect an effect size of 1.6 SD units between treatment conditions using a two-sample t-test with a 0.05 two-sided significance level. Anti-CD38 antibodies statistically significantly reduce inflammation and improve clinical scores (composite of bodyweight, blood on stool, and diarrhea) in DSS animal models.

### Example 5

### Inhibition of CD38 Hydrolase Activity

The ability of various binding compounds in accordance with embodiments of the invention to inhibit CD38 hydrolase activity was assessed. Binding compounds were formulated at a concentration of 0.97 mg/mL in 20 mM Citrate, 100 mM NaCl, pH 6.2. Test substance was stored frozen at −80° C. until the day of use. Cell surface CD38 hydrolase activity was assessed using CD38 positive cell lines Daudi, Ramos, and CHO cells stably transfected to express human CD38. The CD38 positive cell lines were incubated with etheno-NAD substrate in the presence or absence of antibody. Fluorescence at 300 nm excitation and 410 nm emission was measured over time.

Cell Surface CD38 Hydrolase Inhibition Assay: Fluorescence at 300 nm excitation and 410 emission was analyzed over time on the SpectraMax i3x. The untreated RLU was divided by the experimental RLU at a time point prior to saturation determine percent of max CD38 activity.

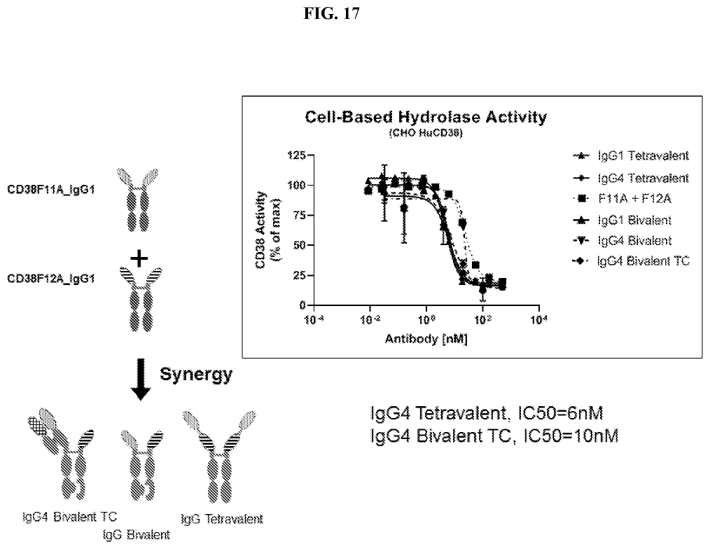

The results are depicted in FIG. 17, and demonstrate that the binding compounds strongly inhibit cell-surface CD38 hydrolase activity on Daudi, Ramos, and CHO cells stably transfected to express human CD38 with EC50 values of 3.4 nM, 5.1 nM, and 9.0 nM, respectively. The max inhibition ranged from 82-88%. These results demonstrate that the binding compounds are strong inhibitors of cell surface CD38 hydrolase activity.

### Example 6

### Activity Summary of Isotype and Valency Formats

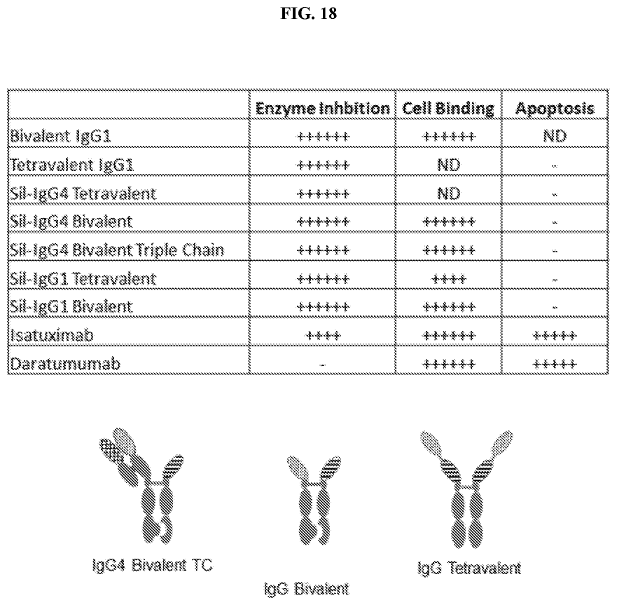

Enzyme inhibition activity, cell binding activity, and apoptosis activity were assessed for various binding compounds in accordance with embodiments of the invention, as well as reference binding compounds isatuximab and daratumumab. The relative levels of these activities were quantified, and are summarized in a tabular format in FIG. 18.

### Example 7

### NAD+ Assay

Studies were performed to assess whether blocking the ecto-NMNase activity of CD38 with the subject binding compounds causes an increase in the NMN-mediated increase in NAD+ within the CD38 expressing B cell lines Ramos and Daudi. The assay is based on the enzymatic cycling reaction in which NAD+ is reduced to NADH. NAD+ reacts with a colorimetric probe that produces a colored product. The intensity of the color is proportional to the NAD+ and NADH within a sample. Oxidized form is selectively destroyed by heating in basic solution, while reduced form is not stable in acidic solution.

Binding compounds were formulated at a concentration of 0.97 mg/mL in 20 mM Citrate, 100 mM NaCl, pH 6.2. Test substance was stored frozen at −80° C. until the day of use.

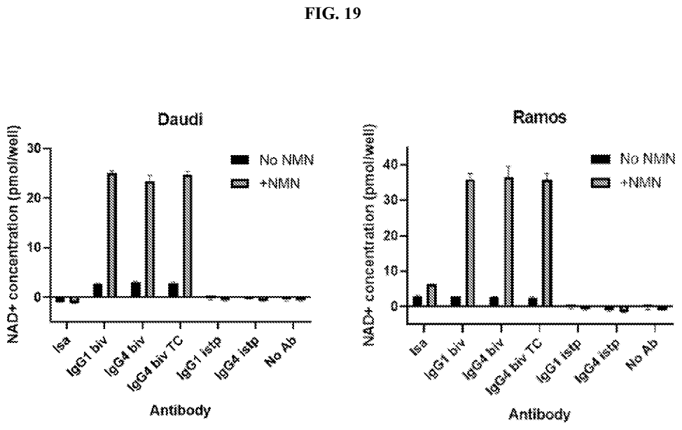

The results are depicted in FIG. 19, and demonstrate that the bispecific, bivalent three chain binding compound remarkably increased the NAD+ levels in the presence of NMN in Daudi or Ramos cells, as compared to the absence of NMN. The results also demonstrate a subtle difference in NAD+ increase in the case of isatuximab in Ramos and not Daudi. This is presumably because isatuximab is also a CD38 enzyme blocker, but it also induces direct apoptosis of cells, Ramos being less sensitive than Daudi. Isatuximab causes direct apoptosis of Daudi cells in 24 hours.

No such increase in NAD+ was observed in isotype-treated cells, or in the absence of any binding compound, demonstrating that the effect of NAD+ increase with or without NMN is completely related to the inhibition of CD38 enzyme activity.

### Example 8

### T-Cell Proliferation in MLR

Various binding compounds in accordance with embodiments of the invention were assessed for their ability to inhibit CD38 hydrolase activity without activating a mixed lymphocyte reaction (MLR). MLR occurs when MHC mismatched immune cells interact, triggering an immune response by T cell hyperproliferation and exacerbated cytokine release. This phenomenon is more pronounced in T cell engaging antibodies or in general, therapeutic antibodies exhibiting effector function. The binding compounds were formulated at a concentration of 0.97 mg/mL in 20 mM Citrate, 100 mM NaCl, pH 6.2. Test substance was stored frozen at −80° C. until the day of use.

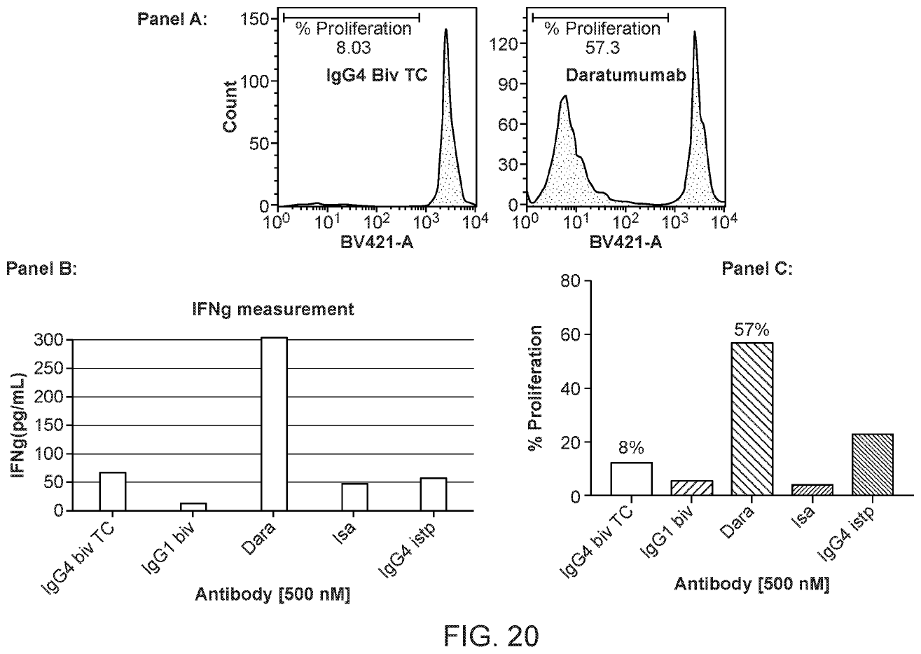

Analyses were performed to assess CD4 T cell proliferation and IFNγ production. The results are depicted in FIG. 20. Panel A demonstrates that the bispecific, bivalent three chain binding compound did not cause an increase in the percentage of CD4 T cell proliferation, while daratumumab did result in an increase in CD4 T cell proliferation. The percentage of CD4 T cell proliferation is also shown in Panel C for a variety of other binding compounds. IFNγ production is shown in Panel B, and demonstrates that daratumumab caused an increase in IFNγ production, while the other binding compounds had no effect on IFNγ production, compared to IgG4 istp control.

The results of this study demonstrate that daratumumab aggravates T cell proliferation and IFNγ production during MLR, whereas the bispecific, bivalent three chain binding compound does not induce T cell activation during MLR.

### Example 9

### Partial Inhibition of Cyclase by IgG4 Bivalent

The ability of a bispecific, bivalent three chain binding compound as depicted in FIG. 10, Panel D, to inhibit CD38 cyclase activity was assessed. The binding compound was formulated at a concentration of 0.97 mg/mL in 20 mM Citrate, 100 mM NaCl, pH 6.2. Test substance was stored frozen at −80° C. until the day of use. Cell surface CD38 cyclase activity was assessed using CD38 positive cell lines Daudi, Ramos, and CHO cells stably transfected to express human CD38. The CD38 positive cell lines were incubated with NGD+ substrate in the presence or absence of the binding compound. Fluorescence at 300 nm excitation and 410 nm emission was measured over time.

Cell Surface CD38 Cyclase Inhibition Assay: Fluorescence at 300nm excitation and 410 emission was analyzed over time on the SpectraMax i3x. The untreated RLU was divided by the experimental RLU at a time point prior to saturation determine percent of max CD38 activity.

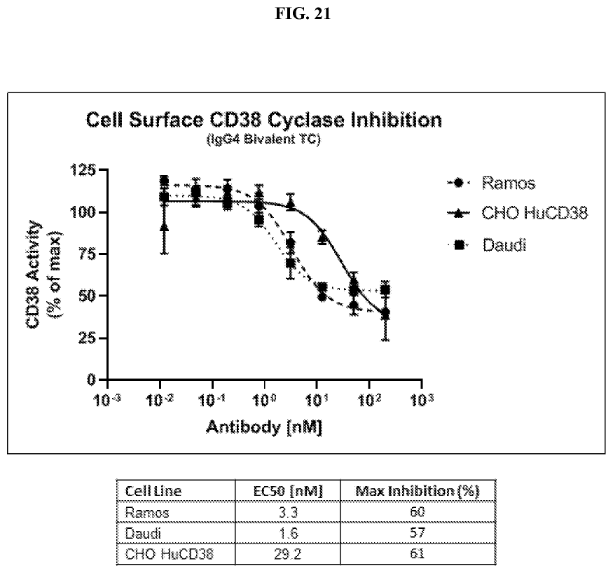

The results are depicted in FIG. 21, and demonstrate that the bispecific, bivalent three chain binding compound partially inhibited CD38 cyclase activity on Ramos, Daudi, and a CHO cell line stably transfected to express human CD38 with EC50 values of 3.3 nM, 1.6 nM, and 29.2 nM, respectively. The max inhibition ranged from 57% to 61%. These results demonstrate that the binding compound is a partial inhibitor of CD38 cyclase activity.

### Example 10

### On Target and Off Target Cell Binding

On target and off target cell binding of a bispecific, bivalent three chain binding compound as depicted in FIG. 11, Panel D, was assessed. The binding compound was formulated at a concentration of 0.97 mg/mL in 20 mM Citrate, 100 mM NaCl, pH 6.2. Test substance was stored frozen at −80° C. until the day of use. Binding to CD38 positive and CD38 negative cell lines was assessed using flow cytometry. The CD38 positive cell lines used were Daudi, Ramos, and a CHO cell line transfected to stably express CD38. The CD38 negative cell lines used were 293-Freestyle, HL-60, K562, and CHO.

Flow Cytometry Analysis of Cell Binding: The average MFI of unstained wells was set as the background signal. To calculate fold over background of each experimental sample, the experimental sample MFI was divided by the average background MFI.

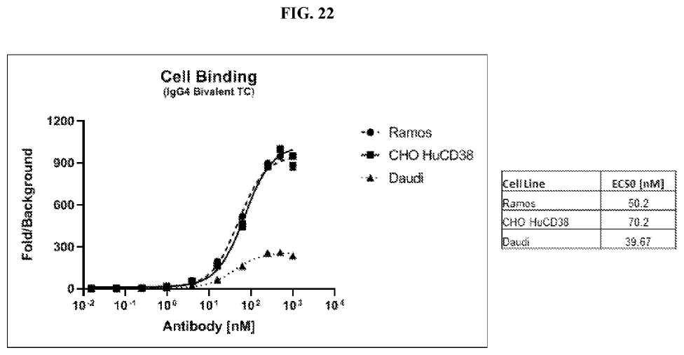

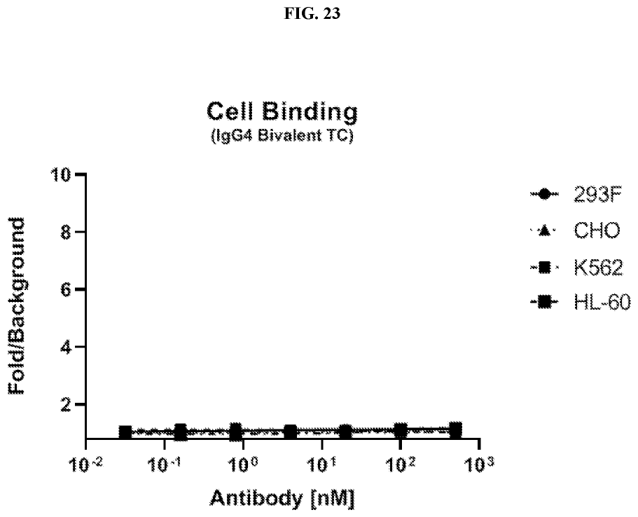

The results of on target cell binding are shown in FIG. 22 and demonstrate that the bispecific, bivalent three chain binding compound binds to Ramos, CHO HuCD38, and Daudi cells with EC50 values of 50.25 nM, 70.2 nM, and 39.67 nM, respectively. The bispecific, bivalent three chain binding compound does not bind to the CD38 negative cell lines tested (293-Freestyle, CHO, K562, and HL-60), as demonstrated in FIG. 23. These results demonstrate that the bispecific, bivalent three chain binding compound binds specifically to CD38, with no binding to off target cell lines.

### Example 11

### Direct Apoptosis

The ability of a bispecific, bivalent three chain binding compound as depicted in FIG. 10, Panel D, to induce direct apoptosis was assessed. The binding compound was formulated at a concentration of 0.97 mg/mL in 20 mM Citrate, 100 mM NaCl, pH 6.2. Test substance was stored frozen at −80° C. until the day of use.

Induction of direct apoptosis was assessed by Annexin-V and 7-AAD staining using flow cytometry. Annexin-V is commonly used to detect apoptotic cells by its ability to bind to phosphatidylserine, a marker of apoptosis when it is present on the outer leaflet of the plasma membrane. 7-AAD binds to double stranded DNA that is taken up by dying or dead cells with compromised membranes. The CD38 positive cell lines used in this study were Daudi and Ramos cells.

Flow Cytometry Analysis of Direct Apoptosis: A quad gate was used to distinguish between early apoptotic (Annexin-V+, 7-AAD-), late apoptotic (Annexin-V+, 7-AAD+), and viable cells (Annexin-V−, 7-AAD-). Concentration of binding compound was plotted against percent viability in Graphpad Prism 8. The resulting plot was fitted to a non-linear regression to determine EC50.

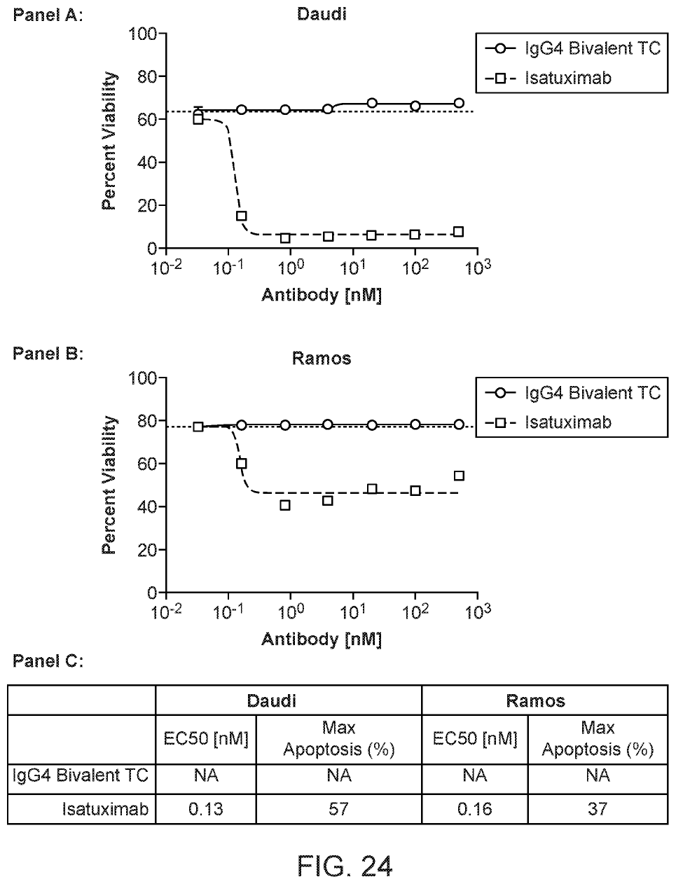

The results are depicted in FIG. 24, and demonstrate that binding of bispecific, bivalent three chain binding compound did not cause direct apoptosis of either Daudi or Ramos cells. Binding of Isatuximab caused direct apoptosis of both Daudi and Ramos cells, with a max apoptosis of 57% and 37%, respectively. These results demonstrate that the bispecific, bivalent three chain binding compound does not cause undesired apoptosis of CD38 positive cells upon binding.

Notwithstanding the appended claims, the disclosure is also defined by the following clauses:

1. A bispecific binding compound comprising: a first polypeptide having binding affinity to a first epitope on an ectoenzyme; and a second polypeptide having binding affinity to a second, non-overlapping epitope on the ectoenzyme.

2. The bispecific binding compound of clause 1, wherein the first polypeptide comprises an antigen-binding domain of a heavy-chain antibody having binding affinity to the first epitope.

3. The bispecific binding compound of clause 2, wherein the second polypeptide comprises an antigen-binding domain of a heavy-chain antibody having binding affinity to the second epitope.

4. The bispecific binding compound of clause 1, wherein the first and second polypeptides each comprise at least a portion of a hinge region.

5. The bispecific binding compound of clause 4, wherein the first and second polypeptides each comprise at least one CH domain.

6. The bispecific binding compound of clause 5, wherein the CH domain comprises a CH2 and/or a CH3 and/or a CH4 domain.

7. The bispecific binding compound of clause 6, wherein the CH domain comprises a CH2 domain and a CH3 domain.

8. The bispecific binding compound of clause 6, wherein the CH domain comprises a CH2 domain, a CH3 domain, and a CH4 domain.

9. The bispecific binding compound of clause 6, wherein the CH domain comprises a human IgG1 Fc region.

10. The bispecific binding compound of clause 9, wherein the human IgG1 Fc region is a silenced human IgG1 Fc region.

11. The bispecific binding compound of clause 6, wherein the CH domain comprises a human IgG4 Fc region.

12. The bispecific binding compound of clause 11, wherein the human IgG4 Fc region is a silenced human IgG4 Fc region.

13. The bispecific binding compound of clause 6, wherein the CH domain does not comprise a CH1 domain.

14. The bispecific binding compound of clause 6, comprising an asymmetric interface between the CH2 and/or the CH3 and/or the CH4 domains of the first and second polypeptides.

15. The bispecific binding compound of clause 1, wherein the first polypeptide comprises: a first antigen-binding domain of a heavy-chain antibody having binding affinity to the first epitope; and a second antigen-binding domain of a heavy-chain antibody having binding affinity to the second epitope.

16. The bispecific binding compound of clause 15, wherein the first and second antigen-binding domains are connected by a polypeptide linker.

17. The bispecific binding compound of clause 16, wherein the polypeptide linker consists of the sequence of SEQ ID NO: 45.

18. The bispecific binding compound of clause 15, wherein the second polypeptide comprises: a first antigen-binding domain of a heavy-chain antibody having binding affinity to the first epitope; and a second antigen-binding domain of a heavy-chain antibody having binding affinity to the second epitope.

19. The bispecific binding compound of clause 18, wherein the first and second antigen-binding domains are connected by a polypeptide linker.

20. The bispecific binding compound of clause 19, wherein the polypeptide linker consists of the sequence of SEQ ID NO: 45.

21. The bispecific binding compound of clause 15, wherein the first and second polypeptides each comprise at least a portion of a hinge region.

22. The bispecific binding compound of clause 21, wherein the first and second polypeptides each comprise at least one CH domain.

23. The bispecific binding compound of clause 22, wherein the CH domain comprise a CH2 and/or a CH3 and/or a CH4 domain.

24. The bispecific binding compound of clause 23, wherein the CH domain comprises a CH2 domain and a CH3 domain.

25. The bispecific binding compound of clause 23, wherein the CH domain comprises a CH2 domain, a CH3 domain, and a CH4 domain.

26. The bispecific binding compound of clause 23, wherein the CH domain does not comprise a CH1 domain.

27. The bispecific binding compound of clause 22, the CH domain comprises a human IgG1 Fc region.

28. The bispecific binding compound of clause 27, wherein the human IgG1 Fc region is a silenced human IgG1 Fc region.

29. The bispecific binding compound of clause 22, the CH domain comprises a human IgG4 Fc region.

30. The bispecific binding compound of clause 29, wherein the human IgG4 Fc region is a silenced human IgG4 Fc region.

31. The bispecific binding compound of clause 1, comprising: a first and a second heavy chain polypeptide, each comprising an antigen-binding domain of a heavy-chain antibody having binding affinity to the first epitope; and a first and a second light chain polypeptide, each comprising an antigen-binding domain of a heavy-chain antibody having binding affinity to the second epitope.

32. The bispecific binding compound of clause 31, wherein the first and second heavy chain polypeptides each comprise at least a portion of a hinge region.

33. The bispecific binding compound of clause 31, wherein the first and second heavy chain polypeptides each comprise at least one CH domain.

34. The bispecific binding compound of clause 33, wherein the CH domain comprises a CH1 and/or a CH2 and/or a CH3 and/or a CH4 domain.

35. The bispecific binding compound of clause 33, wherein the CH domain comprises a CH1 domain and a CH2 domain and a CH3 domain.

36. The bispecific binding compound of clause 33, wherein the CH domain comprises a CH2 domain, a CH3 domain, and a CH4 domain.

37. The bispecific binding compound of clause 31, wherein the first and second light chain polypeptides each comprise a CL domain.

38. The bispecific binding compound of clause 33, the CH domain comprises a human IgG1 Fc region.

39. The bispecific binding compound of clause 38, wherein the human IgG1 Fc region is a silenced human IgG1 Fc region.

40. The bispecific binding compound of clause 33, the CH domain comprises a human IgG4 Fc region.

41. The bispecific binding compound of clause 40, wherein the human IgG4 Fc region is a silenced human IgG4 Fc region.

42. The bispecific binding compound of any one of clauses 1-41, wherein the ectozyme is CD38.

43. The bispecific binding compound of clause 42, wherein: the antigen-binding domain of the heavy-chain antibody having binding affinity to the first epitope or the second epitope on CD38 comprises: (i) a CDR1 sequence having two or fewer substitutions in any of the amino acid sequences of SEQ ID NOs: 1-5; and/or (ii) a CDR2 sequence having two or fewer substitutions in any of the amino acid sequences of SEQ ID NOs: 6-12; and/or (iii) a CDR3 sequence having two or fewer substitutions in any of the amino acid sequences of SEQ ID NOs: 13-17.

44. The bispecific binding compound of clause 43, wherein the CDR1, CDR2, and CDR3 sequences are present in a human framework.

45. The bispecific binding compound of any one of clauses 43-44, comprising: (i) a CDR1 sequence selected from the group consisting of SEQ ID NOs: 1-5; and/or (ii) a CDR2 sequence selected from the group consisting of SEQ ID NOs: 6-12; and/or (iii) a CDR3 sequence selected from the group consisting of SEQ ID NOs: 13-17.

46. The bispecific binding compound of clause 45, comprising: (i) a CDR1 sequence selected from the group consisting of SEQ ID NOs: 1-5; and (ii) a CDR2 sequence selected from the group consisting of SEQ ID NOs: 6-12; and (iii) a CDR3 sequence selected from the group consisting of SEQ ID NOs: 13-17.

47. The bispecific binding compound of clause 46, comprising: a CDR1 sequence of SEQ ID NO: 1, a CDR2 sequence of SEQ ID NO: 6, and a CDR3 sequence of SEQ ID NO: 13; or a CDR1 sequence of SEQ ID NO: 3, a CDR2 sequence of SEQ ID NO: 9, and a CDR3 sequence of SEQ ID NO: 16; or a CDR1 sequence of SEQ ID NO: 4, a CDR2 sequence of SEQ ID NO: 11, and a CDR3 sequence of SEQ ID NO: 17.

48. The bispecific binding compound of clause 47, wherein: the antigen-binding domain of the heavy-chain antibody having binding affinity to the first epitope on CD38 comprises a CDR1 sequence of SEQ ID NO: 1, a CDR2 sequence of SEQ ID NO: 6, and a CDR3 sequence of SEQ ID NO: 13; and the antigen-binding domain of the heavy-chain antibody having binding affinity to the second epitope on CD38 comprises a CDR1 sequence of SEQ ID NO: 3, a CDR2 sequence of SEQ ID NO: 9, and a CDR3 sequence of SEQ ID NO: 16.

49. The bispecific binding compound of any one of clauses 43-48, comprising a variable region sequence having at least 95% sequence identity to any of the sequences of SEQ ID NOs: 18-28.

50. The bispecific binding compounds of clause 49, comprising a variable region sequence selected from the group consisting of SEQ ID NOs: 18-28.

51. The bispecific binding compound of clause 50, wherein: the antigen-binding domain of the heavy-chain antibody having binding affinity to the first epitope on CD38 comprises a variable region sequence of SEQ ID NO: 18; and the antigen-binding domain of the heavy-chain antibody having binding affinity to the second epitope on CD38 comprises a variable region sequence of SEQ ID NO: 23.

52. A heavy-chain antibody that binds to CD38, the heavy-chain antibody comprising an antigen-binding domain comprising: (i) a CDR1 sequence having two or fewer substitutions in any of the amino acid sequences of SEQ ID NOs: 1-5; and/or (ii) a CDR2 sequence having two or fewer substitutions in any of the amino acid sequences of SEQ ID NOs: 6-12; and/or (iii) a CDR3 sequence having two or fewer substitutions in any of the amino acid sequences of SEQ ID NOs: 13-17.

53. The heavy-chain antibody of clause 52, wherein said CDR1, CDR2, and CDR3 sequences are present in a human framework.

54. The heavy-chain antibody of clause 52, further comprising a heavy chain constant region sequence in the absence of a CH1 sequence.

55. The heavy-chain antibody of any one of clauses 52-54, comprising: (a) a CDR1 sequence selected from the group consisting of SEQ ID NOs: 1-5; and/or (b) a CDR2 sequence selected from the group consisting of SEQ ID NOs: 6-12; and/or (c) a CDR3 sequence selected from the group consisting of SEQ ID NOs: 13-17.

56. The heavy-chain antibody of clause 55, comprising: (a) a CDR1 sequence selected from the group consisting of SEQ ID NOs: 1-5; and (b) a CDR2 sequence selected from the group consisting of SEQ ID NOs: 6-12; and (c) a CDR3 sequence selected from the group consisting of SEQ ID NOs: 13-17.

57. The heavy-chain antibody of clause 56, comprising: a CDR1 sequence of SEQ ID NO: 1, a CDR2 sequence of SEQ ID NO: 6, and a CDR3 sequence of SEQ ID NO: 13; or a CDR1 sequence of SEQ ID NO: 3, a CDR2 sequence of SEQ ID NO: 9, and a CDR3 sequence of SEQ ID NO: 16; or a CDR1 sequence of SEQ ID NO: 4, a CDR2 sequence of SEQ ID NO: 11, and a CDR3 sequence of SEQ ID NO: 17.

58. The heavy-chain antibody of any one of clauses 52-57, comprising a variable region sequence having at least 95% sequence identity to any of the sequences of SEQ ID NOs: 18-28.

59. The heavy-chain antibody of clause 58, comprising a variable region sequence selected from the group consisting of SEQ ID NOs: 18-28.

60. The heavy-chain antibody of any one of clauses 52-59, which is monospecific.

61. The heavy-chain antibody of any one of clauses 52-59, which is multi-specific.

62. The heavy-chain antibody of clause 61, which is bispecific.

63. The heavy-chain antibody of clause 62, which has binding affinity to two different epitopes on the same CD38 protein.

64. The heavy-chain antibody of clause 63, wherein the two different epitopes are non-overlapping epitopes.

65. The heavy-chain antibody of clause 61, having binding affinity to an effector cell.

66. The heavy-chain antibody of clause 61, having binding affinity to a T-cell antigen.

67. The heavy-chain antibody of clause 66, having binding affinity to CD3.

68. The heavy-chain antibody of any one of clauses 52-67, which is in a CAR-T format.

69. A bispecific binding compound having binding affinity to a first CD38 epitope and a second, non-overlapping CD38 epitope, the bispecific binding compound comprising: (a) a first polypeptide having binding affinity to the first CD38 epitope comprising: (i) an antigen-binding domain of a heavy-chain antibody comprising a CDR1 sequence of SEQ ID NO: 1, a CDR2 sequence of SEQ ID NO: 6, and a CDR3 sequence of SEQ ID NO: 13; (ii) at least a portion of a hinge region; and (iii) a CH domain comprising a CH2 domain and a CH3 domain; and (b) a second polypeptide having binding affinity to the second CD38 epitope comprising: (i) an antigen-binding domain of a heavy-chain antibody comprising a CDR1 sequence of SEQ ID NO: 3, a CDR2 sequence of SEQ ID NO: 9, and a CDR3 sequence of SEQ ID NO: 16; (ii) at least a portion of a hinge region; and (iii) a CH domain comprising a CH2 domain and a CH3 domain; and (c) an asymmetric interface between the CH3 domain of the first polypeptide and the CH3 domain of the second polypeptide.

70. The bispecific binding compound of clause 69, comprising an Fc region selected from the group consisting of: a human IgG1 Fc region, a human IgG4 Fc region, a silenced human IgG1 Fc region, and a silenced human IgG4 Fc region.

71. A bispecific binding compound having binding affinity to a first CD38 epitope and a second, non-overlapping CD38 epitope, the bispecific binding compound comprising two identical polypeptides, each polypeptide comprising: (i) a first antigen-binding domain of a heavy-chain antibody having binding affinity to the first CD38 epitope, comprising a CDR1 sequence of SEQ ID NO: 1, a CDR2 sequence of SEQ ID NO: 6, and a CDR3 sequence of SEQ ID NO: 13; (ii) a second antigen-binding domain of a heavy-chain antibody having binding affinity to the second CD38 epitope, comprising a CDR1 sequence of SEQ ID NO: 3, a CDR2 sequence of SEQ ID NO: 9, and a CDR3 sequence of SEQ ID NO: 16; (iii) at least a portion of a hinge region; and (iv) a CH domain comprising a CH2 domain and a CH3 domain.

72. The bispecific binding compound of clause 71, comprising an Fc region selected from the group consisting of: a human IgG1 Fc region, a human IgG4 Fc region, a silenced human IgG1 Fc region, and a silenced human IgG4 Fc region.

73. A bispecific binding compound having binding affinity to a first CD38 epitope and a second, non-overlapping CD38 epitope, the bispecific binding compound comprising: (a) a first and a second heavy chain polypeptide, each comprising: (i) an antigen-binding domain of a heavy-chain antibody having binding affinity to the first CD38 epitope, comprising a CDR1 sequence of SEQ ID NO: 1, a CDR2 sequence of SEQ ID NO: 6, and a CDR3 sequence of SEQ ID NO: 13; (ii) at least a portion of a hinge region; and (iii) a CH domain comprising a CH1 domain, a CH2 domain and a CH3 domain; and (b) a first and a second light chain polypeptide, each comprising: (i) an antigen-binding domain of a heavy-chain antibody having binding affinity to the second CD38 epitope, comprising a CDR1 sequence of SEQ ID NO: 3, a CDR2 sequence of SEQ ID NO: 9, and a CDR3 sequence of SEQ ID NO: 16; and (ii) a CL domain.

74. The bispecific binding compound of clause 73, comprising an Fc region selected from the group consisting of: a human IgG1 Fc region, a human IgG4 Fc region, a silenced human IgG1 Fc region, and a silenced human IgG4 Fc region.

75. A pharmaceutical composition comprising a binding compound or a heavy-chain antibody of any one of clauses 1 to 74.

76. A therapeutic combination comprising: the binding compound or heavy-chain antibody according to any one of clauses 52-68; and a second antibody that binds to CD38.

77. The therapeutic combination of clause 76, wherein the second antibody that binds to CD38 is isatuximab or daratumumab.

78. A method for the treatment of a disorder characterized by expression of CD38, comprising administering to a subject with said disorder a binding compound or a heavy-chain antibody of any one of clauses 1 to 74, or a pharmaceutical composition of clause 75.

79. The method of clause 78, wherein the disorder is characterized by a hydrolase enzymatic activity of CD38.

80. The method of clause 78, wherein the disorder is colitis.

81. The method of clause 78, wherein the disorder is multiple myeloma (MM).

82. The method of clause 78, wherein the disorder is an autoimmune disorder.

83. The method of clause 82, wherein the disorder is rheumatoid arthritis (RA).

84. The method of clause 82, wherein the disorder is pemphigus vulgaris (PV).

85. The method of clause 82, wherein the disorder is systemic lupus erythematosus (SLE).

86. The method of clause 82, wherein the disorder is multiple sclerosis (MS), systemic sclerosis or fibrosis.

87. The method of clause 78, wherein the disorder is an ischemic injury.

88. The method of clause 87, wherein the ischemic injury is an ischemic brain injury, an ischemic cardiac injury, an ischemic gastro-intestinal injury, or an ischemic kidney injury.

89. The method of any one of clauses 78-88, further comprising administering to the subject a second antibody that binds to CD38.

90. The method of clause 89, wherein the second antibody that binds to CD38 is isatuximab or daratumumab.

91. A polynucleotide encoding a binding compound or a heavy-chain antibody of any one of clauses 1 to 74.

92. A vector comprising the polynucleotide of clause 91.

93. A cell comprising the vector of clause 92.

94. A method of producing a binding compound or a heavy-chain antibody of any one of clauses 1 to 74, the method comprising growing a cell according to clause 86 under conditions permissive for expression of the binding compound or the heavy-chain antibody, and isolating the binding compound or the heavy-chain antibody from the cell and/or a cell culture medium in which the cell is grown.

95. A method of making a binding compound or a heavy-chain antibody of any one of clauses 1 to 74, the method comprising immunizing a UniRat animal with an ectoenzyme and identifying ectoenzyme-binding heavy chain sequences.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A bispecific binding compound comprising:
a first polypeptide having binding affinity to a first epitope on CD38; and
a second polypeptide having binding affinity to a second, non-overlapping epitope on CD38.

2. The bispecific binding compound of claim 1, wherein the first polypeptide comprises an antigen-binding domain of a heavy-chain antibody having binding affinity to the first epitope or the second epitope on CD38, and comprises:
(i) a CDR1 sequence having two or fewer substitutions in any of the amino acid sequences of SEQ ID NOs: 1-5; and/or
(ii) a CDR2 sequence having two or fewer substitutions in any of the amino acid sequences of SEQ ID NOs: 6-12; and/or
(iii) a CDR3 sequence having two or fewer substitutions in any of the amino acid sequences of SEQ ID NOs: 13-17.

3. The bispecific binding compound of claim 2, wherein the CDR1, CDR2, and CDR3 sequences are present in a human framework.

4. The bispecific binding compound of any one of claims 2-3, comprising:
(i) a CDR1 sequence comprising any one of SEQ ID NOs: 1-5; and/or
(ii) a CDR2 sequence comprising any one of SEQ ID NOs: 6-12; and/or
(iii) a CDR3 sequence comprising any one of SEQ ID NOs: 13-17.

5. The bispecific binding compound of claim 4, comprising:
(i) a CDR1 sequence comprising any one of SEQ ID NOs: 1-5; and
(ii) a CDR2 sequence comprising any one of SEQ ID NOs: 6-12; and
(iii) a CDR3 sequence comprising any one of SEQ ID NOs: 13-17.

6. The bispecific binding compound of claim 5, comprising:
a CDR1 sequence of SEQ ID NO: 1, a CDR2 sequence of SEQ ID NO: 6, and a CDR3 sequence of SEQ ID NO: 13; or
a CDR1 sequence of SEQ ID NO: 3, a CDR2 sequence of SEQ ID NO: 9, and a CDR3 sequence of SEQ ID NO: 16; or
a CDR1 sequence of SEQ ID NO: 4, a CDR2 sequence of SEQ ID NO: 11, and a CDR3 sequence of SEQ ID NO: 17.

7. The bispecific binding compound of claim 6, wherein:
the antigen-binding domain of the heavy-chain antibody having binding affinity to the first epitope on CD38 comprises a CDR1 sequence of SEQ ID NO: 1, a CDR2 sequence of SEQ ID NO: 6, and a CDR3 sequence of SEQ ID NO: 13; and
the antigen-binding domain of the heavy-chain antibody having binding affinity to the second epitope on CD38 comprises a CDR1 sequence of SEQ ID NO: 3, a CDR2 sequence of SEQ ID NO: 9, and a CDR3 sequence of SEQ ID NO: 16.

8. The bispecific binding compound of any one of claims 2-7, comprising a variable region sequence having at least 95% sequence identity to any of the sequences of SEQ ID NOs: 18-28.

9. The bispecific binding compounds of claim 8, comprising a variable region sequence selected from the group consisting of SEQ ID NOs: 18-28.

10. The bispecific binding compound of claim 9, wherein:
the antigen-binding domain of the heavy-chain antibody having binding affinity to the first epitope on CD38 comprises a variable region sequence of SEQ ID NO: 18; and
the antigen-binding domain of the heavy-chain antibody having binding affinity to the second epitope on CD38 comprises a variable region sequence of SEQ ID NO: 23.

11. A heavy-chain antibody that binds to CD38, the heavy-chain antibody comprising an antigen-binding domain comprising:
(i) a CDR1 sequence having two or fewer substitutions in any of the amino acid sequences of SEQ ID NOs: 1-5; and/or
(ii) a CDR2 sequence having two or fewer substitutions in any of the amino acid sequences of SEQ ID NOs: 6-12; and/or
(iii) a CDR3 sequence having two or fewer substitutions in any of the amino acid sequences of SEQ ID NOs: 13-17.

12. The heavy-chain antibody of claim 11, wherein said CDR1, CDR2, and CDR3 sequences are present in a human framework.

13. The heavy-chain antibody of claim 11, further comprising a heavy chain constant region sequence in the absence of a CH1 sequence.

14. The heavy-chain antibody of any one of claims 11-13, comprising:
(a) a CDR1 sequence comprising any one of SEQ ID NOs: 1-5; and/or
(b) a CDR2 sequence comprising any one of SEQ ID NOs: 6-12; and/or
(c) a CDR3 sequence comprising any one of SEQ ID NOs: 13-17.

15. The heavy-chain antibody of claim 14, comprising:
(a) a CDR1 sequence comprising any one of SEQ ID NOs: 1-5; and
(b) a CDR2 sequence comprising any one of SEQ ID NOs: 6-12; and
(c) a CDR3 sequence comprising any one of SEQ ID NOs: 13-17.

16. The heavy-chain antibody of claim 15, comprising:
a CDR1 sequence of SEQ ID NO: 1, a CDR2 sequence of SEQ ID NO: 6, and a CDR3 sequence of SEQ ID NO: 13; or
a CDR1 sequence of SEQ ID NO: 3, a CDR2 sequence of SEQ ID NO: 9, and a CDR3 sequence of SEQ ID NO: 16; or
a CDR1 sequence of SEQ ID NO: 4, a CDR2 sequence of SEQ ID NO: 11, and a CDR3 sequence of SEQ ID NO: 17.

17. The heavy-chain antibody of any one of claims 11-16, comprising a variable region sequence having at least 95% sequence identity to any of the sequences of SEQ ID NOs: 18-28.

18. The heavy-chain antibody of claim 17, comprising a variable region sequence selected from the group consisting of SEQ ID NOs: 18-28.

19. The heavy-chain antibody of any one of claims 11-18, which is monospecific.

20. The heavy-chain antibody of any one of claims 11-18, which is multi-specific.

21. The heavy-chain antibody of claim 20, which is bispecific.

22. The heavy-chain antibody of claim 21, which has binding affinity to two different epitopes on the same CD38 protein.

23. The heavy-chain antibody of claim 22, wherein the two different epitopes are non-overlapping epitopes.

24. The heavy-chain antibody of claim 20, having binding affinity to an effector cell.

25. The heavy-chain antibody of claim 20, having binding affinity to a T-cell antigen.

26. The heavy-chain antibody of claim 25, having binding affinity to CD3.

27. The heavy-chain antibody of any one of claims 11-26, which is in a CAR-T format.

28. A bispecific binding compound having binding affinity to a first CD38 epitope and a second, non-overlapping CD38 epitope, the bispecific binding compound comprising:
(a) a first polypeptide having binding affinity to the first CD38 epitope comprising:
(i) an antigen-binding domain of a heavy-chain antibody comprising a CDR1 sequence of SEQ ID NO: 1, a CDR2 sequence of SEQ ID NO: 6, and a CDR3 sequence of SEQ ID NO: 13;
(ii) at least a portion of a hinge region; and
(iii) a CH domain comprising a CH2 domain and a CH3 domain; and
(b) a second polypeptide having binding affinity to the second CD38 epitope comprising:
(i) an antigen-binding domain of a heavy-chain antibody comprising a CDR1 sequence of SEQ ID NO: 3, a CDR2 sequence of SEQ ID NO: 9, and a CDR3 sequence of SEQ ID NO: 16;
(ii) at least a portion of a hinge region; and
(iii) a CH domain comprising a CH2 domain and a CH3 domain; and
(c) an asymmetric interface between the CH3 domain of the first polypeptide and the CH3 domain of the second polypeptide.

29. The bispecific binding compound of claim 28, comprising an Fc region selected from the group consisting of: a human IgG1 Fc region, a human IgG4 Fc region, a silenced human IgG1 Fc region, and a silenced human IgG4 Fc region.

30. A bispecific binding compound having binding affinity to a first CD38 epitope and a second, non-overlapping CD38 epitope, the bispecific binding compound comprising two identical polypeptides, each polypeptide comprising:
(i) a first antigen-binding domain of a heavy-chain antibody having binding affinity to the first CD38 epitope, comprising a CDR1 sequence of SEQ ID NO: 1, a CDR2 sequence of SEQ ID NO: 6, and a CDR3 sequence of SEQ ID NO: 13;
(ii) a second antigen-binding domain of a heavy-chain antibody having binding affinity to the second CD38 epitope, comprising a CDR1 sequence of SEQ ID NO: 3, a CDR2 sequence of SEQ ID NO: 9, and a CDR3 sequence of SEQ ID NO: 16;
(iii) at least a portion of a hinge region; and
(iv) a CH domain comprising a CH2 domain and a CH3 domain.

31. The bispecific binding compound of claim 30, comprising an Fc region selected from the group consisting of: a human IgG1 Fc region, a human IgG4 Fc region, a silenced human IgG1 Fc region, and a silenced human IgG4 Fc region.

32. A bispecific binding compound having binding affinity to a first CD38 epitope and a second, non-overlapping CD38 epitope, the bispecific binding compound comprising:
(a) a first and a second heavy chain polypeptide, each comprising:
(i) an antigen-binding domain of a heavy-chain antibody having binding affinity to the first CD38 epitope, comprising a CDR1 sequence of SEQ ID NO: 1, a CDR2 sequence of SEQ ID NO: 6, and a CDR3 sequence of SEQ ID NO: 13;
(ii) at least a portion of a hinge region; and
(iii) a CH domain comprising a CH1 domain, a CH2 domain and a CH3 domain; and
(b) a first and a second light chain polypeptide, each comprising:
(i) an antigen-binding domain of a heavy-chain antibody having binding affinity to the second CD38 epitope, comprising a CDR1 sequence of SEQ ID NO: 3, a CDR2 sequence of SEQ ID NO: 9, and a CDR3 sequence of SEQ ID NO: 16; and
(ii) a CL domain.

33. The bispecific binding compound of claim 32, comprising an Fc region selected from the group consisting of: a human IgG1 Fc region, a human IgG4 Fc region, a silenced human IgG1 Fc region, and a silenced human IgG4 Fc region.

34. A bispecific binding compound having binding affinity to a first CD38 epitope and a second, non-overlapping CD38 epitope, the bispecific binding compound comprising:
(a) a first polypeptide subunit comprising a heavy chain variable region comprising a CDR1 sequence of SEQ ID NO: 1, a CDR2 sequence of SEQ ID NO: 6, and a CDR3 sequence of SEQ ID NO: 13 in a human heavy chain framework;
(b) a second polypeptide subunit comprising a light chain variable region comprising a CDR1 sequence of SEQ ID NO: 49, a CDR2 sequence of SEQ ID NO: 50, and a CDR3 sequence of SEQ ID NO: 51, in a human light chain framework;
wherein the first polypeptide subunit and the second polypeptide subunit together have binding affinity to the first CD38 epitope; and
(c) a third polypeptide subunit comprising an antigen-binding domain of a heavy-chain antibody comprising a CDR1 sequence of SEQ ID NO: 3, a CDR2 sequence of SEQ ID NO: 9, and a CDR3 sequence of SEQ ID NO: 16 in a human heavy chain framework, in a monovalent or bivalent configuration;
wherein the third polypeptide subunit has binding affinity to the second, non-overlapping CD38 epitope.

35. The bispecific binding compound of claim 34, wherein the first polypeptide subunit further comprises a CH1 domain, at least a portion of a hinge region, a CH2 domain, and a CH3 domain.

36. The bispecific binding compound of claim 34 or 35, wherein the third polypeptide subunit further comprises a constant region sequence comprising at least a portion of a hinge region, a CH2 domain, and a CH3 domain, in the absence of a CH1 domain.

37. The bispecific binding compound of any one of claims 34-36, wherein the human light chain framework is a human kappa light chain framework or a human lambda light chain framework.

38. The bispecific binding compound of any one of claims 34-37, wherein the second polypeptide subunit further comprises a CL domain.

39. The bispecific binding compound of any one of claims 34-38, comprising an Fc region selected from the group consisting of: a human IgG1 Fc region, a human IgG4 Fc region, a silenced human IgG1 Fc region, and a silenced human IgG4 Fc region.

40. The bispecific binding compound of any one of claims 34-39, comprising an asymmetric interface between the CH3 domain of the first polypeptide subunit and the CH3 domain of the third polypeptide subunit.

41. A bispecific binding compound having binding affinity to a first CD38 epitope and a second, non-overlapping CD38 epitope, comprising:
(a) a first heavy chain polypeptide comprising the sequence of SEQ ID NO: 46;
(b) a first light chain polypeptide comprising the sequence of SEQ ID NO: 48; and
(c) a second heavy chain polypeptide comprising the sequence of SEQ ID NO: 47.

42. A pharmaceutical composition comprising a binding compound or a heavy-chain antibody of any one of claims 1 to 41.

43. A method for the treatment of a disorder characterized by expression of CD38, comprising administering to a subject with said disorder a binding compound or a heavy-chain antibody of any one of claims 1 to 41, or a pharmaceutical composition of claim 42.

44. The method of claim 43, wherein the disorder is characterized by a hydrolase enzymatic activity of CD38.

45. The method of claim 43, wherein the disorder is colitis.

46. The method of claim 43, wherein the disorder is multiple myeloma (MM).

47. The method of claim 43, wherein the disorder is an autoimmune disorder.

48. The method of claim 47, wherein the disorder is rheumatoid arthritis (RA).

49. The method of claim 47, wherein the disorder is pemphigus vulgaris (PV).

50. The method of claim 47, wherein the disorder is systemic lupus erythematosus (SLE).

51. The method of claim 47, wherein the disorder is multiple sclerosis (MS), systemic sclerosis or fibrosis.

52. The method of claim 43, wherein the disorder is an ischemic injury.

53. The method of claim 52, wherein the ischemic injury is an ischemic brain injury, an ischemic cardiac injury, an ischemic gastro-intestinal injury, or an ischemic kidney injury.

54. The method of any one of claims 43-53, further comprising administering to the subject a second antibody that binds to CD38.

55. The method of claim 54, wherein the second antibody that binds to CD38 is isatuximab or daratumumab.

56. A polynucleotide encoding a binding compound or a heavy-chain antibody of any one of claims 1 to 41.

57. A vector comprising the polynucleotide of claim 56.

58. A cell comprising the vector of claim 57.

59. A method of producing a binding compound or a heavy-chain antibody of any one of claims 1 to 41, the method comprising growing a cell according to claim 58 under conditions permissive for expression of the binding compound or the heavy-chain antibody, and isolating the binding compound or the heavy-chain antibody from the cell and/or a cell culture medium in which the cell is grown.

60. A method of making a binding compound or a heavy-chain antibody of any one of claims 1 to 41, the method comprising immunizing a UniRat animal with a CD38 protein and identifying CD38 protein-binding heavy chain sequences.


SEQUENCE LISTING (53 sequences)

SEQ ID NO: 1 - protein, 8 aa, Artificial Sequence
  source: /note=“Description of Artificial Sequence Synthetic peptide”
GFTFSSYG

SEQ ID NO: 2 - protein, 8 aa, Artificial Sequence
  source: /note=“Description of Artificial Sequence Synthetic peptide”
GFTFSGYG

SEQ ID NO: 3 - protein, 8 aa, Artificial Sequence
  source: /note=“Description of Artificial Sequence Synthetic peptide”
GFTFSSSW

SEQ ID NO: 4 - protein, 10 aa, Artificial Sequence
  source: /note=“Description of Artificial Sequence Synthetic peptide”
GGSISSSLFY

SEQ ID NO: 5 - protein, 10 aa, Artificial Sequence
  source: /note=“Description of Artificial Sequence Synthetic peptide”
GGSISSSNYH

SEQ ID NO: 6 - protein, 8 aa, Artificial Sequence
  source: /note=“Description of Artificial Sequence Synthetic peptide”
ISDDGSNK

SEQ ID NO: 7 - protein, 8 aa, Artificial Sequence
  source: /note=“Description of Artificial Sequence Synthetic peptide”
ISYDGSKK

SEQ ID NO: 8 - protein, 8 aa, Artificial Sequence
  source: /note=“Description of Artificial Sequence Synthetic peptide”
ISYDGSNK

SEQ ID NO: 9 - protein, 8 aa, Artificial Sequence
  source: /note=“Description of Artificial Sequence Synthetic peptide”
IKQDGSEK

SEQ ID NO: 10 - protein, 8 aa, Artificial Sequence
  source: /note=“Description of Artificial Sequence Synthetic peptide”
INQDGSEK

SEQ ID NO: 11 - protein, 7 aa, Artificial Sequence
  source: /note=“Description of Artificial Sequence Synthetic peptide”
IHDSGST

SEQ ID NO: 12 - protein, 7 aa, Artificial Sequence
  source: /note=“Description of Artificial Sequence Synthetic peptide”
IYYSGST

SEQ ID NO: 13 - protein, 17 aa, Artificial Sequence
  source: /note=“Description of Artificial Sequence Synthetic peptide”
AKDRGTMRVVVYDTLDI

SEQ ID NO: 14 - protein, 17 aa, Artificial Sequence
  source: /note=“Description of Artificial Sequence Synthetic peptide”
AKDRGTMRVAVYDAFDL

SEQ ID NO: 15 - protein, 17 aa, Artificial Sequence
  source: /note=“Description of Artificial Sequence Synthetic peptide”
AKDRGTMRVAVYDTLDI

SEQ ID NO: 16 - protein, 11 aa, Artificial Sequence
  source: /note=“Description of Artificial Sequence Synthetic peptide”
ARDRRGPFFHI

SEQ ID NO: 17 - protein, 17 aa, Artificial Sequence
  source: /note=“Description of Artificial Sequence Synthetic peptide”
ARGPRGFYSSGPDDFDI

SEQ ID NO: 18 - protein, 124 aa, Artificial Sequence
  source: /note=“Description of Artificial Sequence Synthetic polypeptide”
QVQLVESGGGVVQPGRSLRLSCAASGFTFSSYGMHWVRQAPGKEREWVAVISDDGSNKYYADSVKGRFTISRDNSKNTLYLQMNSLRVEDTAVYYCAKDRGTMRVVVYDTLDIWGQGTLVTVSS

SEQ ID NO: 19 - protein, 124 aa, Artificial Sequence
  source: /note=“Description of Artificial Sequence Synthetic polypeptide”
EVQLLESGGGVVQPGRSLRLSCAASGFTFSSYGMHWVRQAPGKEREWVAVISDDGSNKYYADSVKGRFTISRDNSKNTLYLQMNSLRAEDTAVYYCAKDRGTMRVAVYDAFDLWGQGTLVTVSS

SEQ ID NO: 20 - protein, 124 aa, Artificial Sequence
  source: /note=“Description of Artificial Sequence Synthetic polypeptide”
QVQLVESGGGVVQPGRSLTLSCAASGFTFSSYGMHWVRQAPGKEREWVAVISDDGSNKYYADSVKGRFTISRDNSKNTLYLQMNSLRVEDTAVYYCAKDRGTMRVAVYDTLDIWGQGTLVTVSS

SEQ ID NO: 21 - protein, 124 aa, Artificial Sequence
  source: /note=“Description of Artificial Sequence Synthetic polypeptide”
QVQLVESGGGVVQPGRSLRLSCAASGFTFSSYGMHWVRQAPGKEREWVAVISYDGSKKYYADSVKGRFTISRDNSKNTLYLQMNSLRVEDTAVYYCAKDRGTMRVVVYDTLDIWGQGTLVTVSS

SEQ ID NO: 22 - protein, 124 aa, Artificial Sequence
  source: /note=“Description of Artificial Sequence Synthetic polypeptide”
QVQLVESGGGLVQPGGSLRLSCAASGFTFSGYGMHWLRQAPGKEREWVAVISYDGSNKYYADSVKGRFTISRDNSKNTLYLQMNSLRVEDTAVYYCAKDRGTMRVVVYDTLDIWGQGTLVTVSS

SEQ ID NO: 23 - protein, 118 aa, Artificial Sequence
  source: /note=“Description of Artificial Sequence Synthetic polypeptide”
GVQLVESGGGLVQPGGSLRLSCTASGFTFSSSWMSWVRQAPGKGLEWVANIKQDGSEKDYVDSAKGRFTISRDNAKNSLYLQMNNLRAEDTAVYYCARDRRGPFFHIWGQGTLVTVSS

SEQ ID NO: 24 - protein, 118 aa, Artificial Sequence
  source: /note=“Description of Artificial Sequence Synthetic polypeptide”
GVQLVESGGGLVQPGGSLRLSCAASGFTFSSSWMSWVRQAPGKGLEWVANIKQDGSEKDYVDSAKGRFTISRDNAKNSLYLQMNNLRAEDTAVYYCARDRRGPFFHIWGQGTLVTVSS

SEQ ID NO: 25 - protein, 118 aa, Artificial Sequence
  source: /note=“Description of Artificial Sequence Synthetic polypeptide”
GVQLVESGGGLVQPGGSLRLSCAASGFTFSSSWMSWVRQAPGKGLEWVANINQDGSEKDYVDSAKGRFTISRDNAKNSLYLQMNSLRVEDTAVYYCARDRRGPFFHIWGQGTLVTVSS

SEQ ID NO: 26 - protein, 118 aa, Artificial Sequence
  source: /note=“Description of Artificial Sequence Synthetic polypeptide”
GVQLVESGGGLVQPGGSLRLSCAASGFTFSSSWMSWVRQAPGKGLEWVANIKQDGSEKDYVDSAKGRFTISRDNAKNSLYLQMNSLTAEDTAVYYCARDRRGPFFHIWGQGTLVTVSS

SEQ ID NO: 27 - protein, 125 aa, Artificial Sequence
  source: /note=“Description of Artificial Sequence Synthetic polypeptide”
QLQLQESGPGLVKPSETLSLTCTVSGGSISSSLFYWGWIRQPPGKGLEWIGSIHDSGSTYYNPSLKSRVTISADTSKNQFSLKLNSVTATDTAEYYCARGPRGFYSSGPDDFDIWGQGTLVTVSS

SEQ ID NO: 28 - protein, 125 aa, Artificial Sequence
  source: /note=“Description of Artificial Sequence Synthetic polypeptide”
QLQLQESGPGLVKSSETLSLTCIVSGGSISSSNYHWGWSRQPPGKGQEWIGSIYYSGSTYYNPSLKSRVTISGDTSKNQFSLKLSSVTAADTAVYYCARGPRGFYSSGPDDFDIWGQGTLVTVSS

SEQ ID NO: 29 - protein, 5 aa, Artificial Sequence
  source: /note=“Description of Artificial Sequence Synthetic peptide”
GGGGS

SEQ ID NO: 30 - protein, 450 aa, Artificial Sequence
  source: /note=“Description of Artificial Sequence Synthetic polypeptide”
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, Artificial Sequence
  source: /note=“Description of Artificial Sequence Synthetic polypeptide”
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 372 bp, Artificial Sequence
  source: /note=“Description of Artificial Sequence Synthetic polynucleotide”
caggtgcagctggtggagtcggggggaggcgtggtccagcctgggaggtccctgagactctcctgtgcagcctctggattcaccttcagtagctatggcatgcactgggtccgccaggctccaggcaaggagcgggagtgggtggcagttatatcagatgatggaagtaataaatattatgcagactccgtgaagggccgattcaccatctccagagacaattccaagaacacgctgtatctccaaatgaacagcctgagagttgaggacacggctgtgtattactgtgcgaaagatcggggtactatgagagtagtggtttatgatactttggatatctggggccagggcaccctggtcaccgtctcctca

SEQ ID NO: 33 - DNA, 372 bp, Artificial Sequence
  source: /note=“Description of Artificial Sequence Synthetic polynucleotide”
gaggtgcagctgttggagtctgggggaggcgtggtccagcctgggaggtccctgagactctcttgtgcagcctctggattcaccttcagtagctatggcatgcactgggtccgccaggctccaggcaaggagcgggagtgggtggcagttatatcagatgatggaagtaataaatactatgcagactccgtgaagggccgattcaccatctccagagacaattccaagaacacgctgtatctgcaaatgaacagcctgagagctgaggacacggctgtgtattactgtgcgaaagatcggggtactatgagagtagcggtttatgatgcttttgatctctggggccagggcaccctggtcaccgtctcctca

SEQ ID NO: 34 - DNA, 372 bp, Artificial Sequence
  source: /note=“Description of Artificial Sequence Synthetic polynucleotide”
caggtgcagctggtggagtcggggggaggcgtggtccagcctgggaggtccctgacactctcctgtgcagcctctggattcaccttcagtagctatggcatgcactgggtccgccaggctccaggcaaggagcgggagtgggtggcagttatatcagatgatggaagtaataaatattatgcagactccgtgaagggccgattcaccatctccagagacaattccaagaacacgctgtatctgcaaatgaacagcctgagagttgaggacacggctgtgtattactgtgcgaaagatcggggtactatgagagtagcggtttatgatactttggatatctggggccagggcaccctggtcaccgtctcctca

SEQ ID NO: 35 - DNA, 372 bp, Artificial Sequence
  source: /note=“Description of Artificial Sequence Synthetic polynucleotide”
caggtgcagctggtggagtctgggggaggcgtggtccagcctgggaggtccctgagactctcctgtgcagcctctggattcaccttcagtagctatggcatgcactgggtccgccaggctccaggcaaggagcgggagtgggtggcagttatatcatatgatggaagtaagaaatactatgcagactccgtgaagggccgattcaccatctccagagacaattccaagaacacgctgtatctccaaatgaacagcctgagagttgaggacacggctgtgtattactgtgcgaaagatcggggtactatgagagtagtggtttatgatactttggatatctggggccagggcaccctggtcaccgtctcctca

SEQ ID NO: 36 - DNA, 372 bp, Artificial Sequence
  source: /note=“Description of Artificial Sequence Synthetic polynucleotide”
caggtgcagctggtggagtctgggggaggcttggtacagcctggggggtccctgagactctcctgtgcagcctctggattcaccttcagtggctatggcatgcactggctccgccaggctccaggcaaggagcgggagtgggtggcagttatatcatatgatggaagtaataaatactatgcagactccgtgaagggccgattcaccatctccagagacaattccaagaacacgctgtatctccaaatgaacagcctgagagttgaggacacggctgtgtattactgtgcgaaagatcggggtactatgagagtagtggtttatgatactttggatatctggggccagggcaccctggtcaccgtctcctca

SEQ ID NO: 37 - DNA, 354 bp, Artificial Sequence
  source: /note=“Description of Artificial Sequence Synthetic polynucleotide”
ggggtgcagctggtggagtctgggggaggcttggtccagcctggggggtccctgagactctcctgtacagcctctggattcacctttagtagctcttggatgagctgggtccgccaggctccagggaaggggctggaatgggtggccaacataaagcaagatggaagtgagaaagactatgtggactctgcgaagggccgattcaccatctccagagacaacgccaagaactcactgtatctgcaaatgaacaacctgagagccgaggacacggctgtgtattactgtgcgagagataggagggggcccttttttcatatctggggccagggcaccctggtcaccgtctcctca

SEQ ID NO: 38 - DNA, 354 bp, Artificial Sequence
  source: /note=“Description of Artificial Sequence Synthetic polynucleotide”
ggggtgcagctggtggagtctgggggaggcttggtccagcctggggggtccctgagactctcctgtgcagcctctggattcacctttagtagctcttggatgagctgggtccgccaggctccagggaaggggctggaatgggtggccaacataaagcaagatggaagtgagaaagactatgtggactctgcgaagggccgattcaccatctccagagacaacgccaagaactcactgtatctgcaaatgaacaacctgagagccgaggacacggctgtgtattactgtgcgagagataggagggggcccttttttcatatctggggccagggcaccctggtcaccgtctcctca

SEQ ID NO: 39 - DNA, 354 bp, Artificial Sequence
  source: /note=“Description of Artificial Sequence Synthetic polynucleotide”
ggggtgcagctggtggagtctgggggaggcttggtccagcctggggggtccctgagactctcctgtgcagcctctggattcacctttagtagctcttggatgagctgggtccgccaggctccagggaaggggctggaatgggtggccaacataaaccaagatggaagtgagaaagactatgtggactctgcgaagggccgattcaccatctccagagacaacgccaagaactcactgtatctgcaaatgaacagcctgagagtcgaggacacggctgtgtattactgtgcgagagataggagggggcccttttttcatatctggggccagggcaccctggtcaccgtctcctca

SEQ ID NO: 40 - DNA, 354 bp, Artificial Sequence
  source: /note=“Description of Artificial Sequence Synthetic polynucleotide”
ggggtgcagctggtggagtctgggggaggcttggtccagcctggggggtccctgagactctcctgtgcagcctctggattcacctttagtagctcttggatgagctgggtccgccaggctccagggaaggggctggaatgggtggccaacataaagcaagatggaagtgagaaagactatgtggactctgcgaagggccgattcaccatctccagagacaacgccaagaactcactgtatctgcaaatgaacagcctgacagccgaggacacggccgtgtattactgtgcgagagataggagggggcccttttttcatatctggggccagggcaccctggtcaccgtctcctca

SEQ ID NO: 41 - DNA, 375 bp, Artificial Sequence
  source: /note=“Description of Artificial Sequence Synthetic polynucleotide”
cagctgcagctgcaggagtcgggcccaggactggtgaagccttcggagaccctgtccctcacctgcactgtctctggtggctccatcagtagtagtcttttctactgggggtggatccgccagcccccggggaaggggctggagtggattgggagtatccatgatagtgggagcacctactacaacccgtccctcaagagtcgagtcaccatatccgcagacacgtccaagaaccagttctccctgaagctgaactctgtgaccgccacagacacggctgagtattactgtgcgagagggccgcgcggtttctatagcagtggccctgatgattttgatatctggggccagggcaccctggtcaccgtctcctca

SEQ ID NO: 42 - DNA, 375 bp, Artificial Sequence
  source: /note=“Description of Artificial Sequence Synthetic polynucleotide”
cagctgcagctgcaggagtcgggcccaggactggtgaagtcttcggagaccctgtccctcacctgcattgtctctggtggctccatcagcagtagtaattaccactggggctggagccgccagcccccagggaaggggcaggagtggatcgggagtatctattacagtggaagtacctactacaacccgtccctcaagagtcgagtcaccatttccggagacacgtccaagaaccagttctccctgaagctgagctctgtgaccgccgcagacacggctgtgtattactgtgcgagagggccgcgcggtttctatagcagtggccctgatgattttgatatctggggccagggcaccctggtcaccgtctcctca

SEQ ID NO: 43 - protein, 330 aa, Homo sapiens [sequence omitted]

SEQ ID NO: 44 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 10 aa, Artificial Sequence
  source: /note=“Description of Artificial Sequence Synthetic peptide”
GGGGSGGGGS

SEQ ID NO: 46 - protein, 451 aa, Artificial Sequence
  source: /note=“Description of Artificial Sequence Synthetic polypeptide”
QVQLVESGGGVVQPGRSLRLSCAASGFTFSSYGMHWVRQAPGKEREWVAVISDDGSNKYYADSVKGRFTISRDNSKNTLYLQMNSLRVEDTAVYYCAKDRGTMRVVVYDTLDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 347 aa, Artificial Sequence
  source: /note=“Description of Artificial Sequence Synthetic polypeptide”
GVQLVESGGGLVQPGGSLRLSCTASGFTFSSSWMSWVRQAPGKGLEWVANIKQDGSEKDYVDSAKGRFTISRDNAKNSLYLQMNNLRAEDTAVYYCARDRRGPFFHIWGQGTLVTVSSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 48 - protein, 214 aa, Artificial Sequence
  source: /note=“Description of Artificial Sequence Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 6 aa, Artificial Sequence
  source: /note=“Description of Artificial Sequence Synthetic peptide”
QSVSSN

SEQ ID NO: 50 - protein, 3 aa, Artificial Sequence
  source: /note=“Description of Artificial Sequence Synthetic peptide”
GAS

SEQ ID NO: 51 - protein, 9 aa, Artificial Sequence
  source: /note=“Description of Artificial Sequence Synthetic peptide”
QQYNNWPWT

SEQ ID NO: 52 - protein, 107 aa, Artificial Sequence
  source: /note=“Description of Artificial Sequence Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 53 - protein, 50 aa, Artificial Sequence
  source: /note=“Description of Artificial Sequence Synthetic polypeptide”
  SITE (1)..(50): /note=“This sequence may encompass 1-10 ′Gly Gly Gly Gly Ser′ repeating units”
  source: /note=“See specification as filed for detailed description of substitutions and preferred embodiments”
GGGGSGGGGSGGGGSGGGGSGGGGSGGGGSGGGGSGGGGSGGGGSGGGGS